SEQUENCE LISTING (79 sequences)

SEQ ID NO: 1 - protein, 3 aa, Artificial Sequence
  Description of Artificial Sequencesynthetic peptide
KSP

SEQ ID NO: 2 - protein, 4 aa, Artificial Sequence
  Description of Artificial Sequencesynthetic peptide
AKSP

SEQ ID NO: 3 - protein, 5 aa, Artificial Sequence
  Description of Artificial Sequencesynthetic peptide
AKSPA

SEQ ID NO: 4 - protein, 6 aa, Artificial Sequence
  Description of Artificial Sequencesynthetic peptide
AKSPEK

SEQ ID NO: 5 - protein, 8 aa, Artificial Sequence
  Description of Artificial Sequencesynthetic peptide
AKSPVKEE

SEQ ID NO: 6 - protein, 16 aa, Artificial Sequence
  Description of Artificial Sequencesynthetic peptide
AKSPAEAKSPAEAKSP

SEQ ID NO: 7 - protein, 16 aa, Artificial Sequence
  Description of Artificial Sequencesynthetic peptide
AKSPAEAKSPEKAKSP

SEQ ID NO: 8 - protein, 18 aa, Artificial Sequence
  Description of Artificial Sequencesynthetic peptide
AKSPAEAKSPVKEEAKSP

SEQ ID NO: 9 - protein, 16 aa, Artificial Sequence
  Description of Artificial Sequencesynthetic peptide
AKSPEKAKSPAEAKSP

SEQ ID NO: 10 - protein, 16 aa, Artificial Sequence
  Description of Artificial Sequencesynthetic peptide
AKSPEKAKSPEKAKSP

SEQ ID NO: 11 - protein, 18 aa, Artificial Sequence
  Description of Artificial Sequencesynthetic peptide
AKSPEKAKSPVKEEAKSP

SEQ ID NO: 12 - protein, 18 aa, Artificial Sequence
  Description of Artificial Sequencesynthetic peptide
AKSPVKEEAKSPAEAKSP

SEQ ID NO: 13 - protein, 18 aa, Artificial Sequence
  Description of Artificial Sequencesynthetic peptide
AKSPVKEEAKSPEKAKSP

SEQ ID NO: 14 - protein, 16 aa, Artificial Sequence
  Description of Artificial Sequencesynthetic peptide
AKSPAEAKSPAEAKSP

SEQ ID NO: 15 - protein, 16 aa, Artificial Sequence
  Description of Artificial Sequencesynthetic peptide
AKSPAEAKSPAEAKSP

SEQ ID NO: 16 - protein, 16 aa, Artificial Sequence
  Description of Artificial Sequencesynthetic peptide
AKSPAEAKSPAEAKSP

SEQ ID NO: 17 - protein, 16 aa, Artificial Sequence
  Description of Artificial Sequencesynthetic peptide
AKSPAEAKSPAEAKSP

SEQ ID NO: 18 - protein, 16 aa, Artificial Sequence
  Description of Artificial Sequencesynthetic peptide
AKSPAEAKSPAEAKSP

SEQ ID NO: 19 - protein, 16 aa, Artificial Sequence
  Description of Artificial Sequencesynthetic peptide
AKSPAEAKSPAEAKSP

SEQ ID NO: 20 - protein, 16 aa, Artificial Sequence
  Description of Artificial Sequencesynthetic peptide
AKSPAEAKSPAEAKSP

SEQ ID NO: 21 - protein, 17 aa, Artificial Sequence
  Description of Artificial Sequencesynthetic peptide
AKSPAEAKSPAEAKSPA

SEQ ID NO: 22 - protein, 16 aa, Artificial Sequence
  Description of Artificial Sequencesynthetic peptide
AKSPAEAKSPEKAKSP

SEQ ID NO: 23 - protein, 16 aa, Artificial Sequence
  Description of Artificial Sequencesynthetic peptide
AKSPAEAKSPEKAKSP

SEQ ID NO: 24 - protein, 16 aa, Artificial Sequence
  Description of Artificial Sequencesynthetic peptide
AKSPAEAKSPEKAKSP

SEQ ID NO: 25 - protein, 16 aa, Artificial Sequence
  Description of Artificial Sequencesynthetic peptide
AKSPAEAKSPEKAKSP

SEQ ID NO: 26 - protein, 16 aa, Artificial Sequence
  Description of Artificial Sequencesynthetic peptide
AKSPAEAKSPEKAKSP

SEQ ID NO: 27 - protein, 16 aa, Artificial Sequence
  Description of Artificial Sequencesynthetic peptide
AKSPAEAKSPEKAKSP

SEQ ID NO: 28 - protein, 16 aa, Artificial Sequence
  Description of Artificial Sequencesynthetic peptide
AKSPAEAKSPEKAKSP

SEQ ID NO: 29 - protein, 17 aa, Artificial Sequence
  Description of Artificial Sequencesynthetic peptide
AKSPAEAKSPEKAKSPA

SEQ ID NO: 30 - protein, 18 aa, Artificial Sequence
  Description of Artificial Sequencesynthetic peptide
AKSPAEAKSPVKEEAKSP

SEQ ID NO: 31 - protein, 18 aa, Artificial Sequence
  Description of Artificial Sequencesynthetic peptide
AKSPAEAKSPVKEEAKSP

SEQ ID NO: 32 - protein, 18 aa, Artificial Sequence
  Description of Artificial Sequencesynthetic peptide
AKSPAEAKSPVKEEAKSP

SEQ ID NO: 33 - protein, 18 aa, Artificial Sequence
  Description of Artificial Sequencesynthetic peptide
AKSPAEAKSPVKEEAKSP

SEQ ID NO: 34 - protein, 18 aa, Artificial Sequence
  Description of Artificial Sequencesynthetic peptide
AKSPAEAKSPVKEEAKSP

SEQ ID NO: 35 - protein, 18 aa, Artificial Sequence
  Description of Artificial Sequencesynthetic peptide
AKSPAEAKSPVKEEAKSP

SEQ ID NO: 36 - protein, 19 aa, Artificial Sequence
  Description of Artificial Sequencesynthetic peptide
AKSPAEAKSPVKEEAKSPV

SEQ ID NO: 37 - protein, 18 aa, Artificial Sequence
  Description of Artificial Sequencesynthetic peptide
AKSPAEAKSPVKEEAKSP

SEQ ID NO: 38 - protein, 19 aa, Artificial Sequence
  Description of Artificial Sequencesynthetic peptide
AKSPAEAKSPVKEEAKSPV

SEQ ID NO: 39 - protein, 16 aa, Artificial Sequence
  Description of Artificial Sequencesynthetic peptide
AKSPEKAKSPAEAKSP

SEQ ID NO: 40 - protein, 16 aa, Artificial Sequence
  Description of Artificial Sequencesynthetic peptide
AKSPEKAKSPAEAKSP

SEQ ID NO: 41 - protein, 16 aa, Artificial Sequence
  Description of Artificial Sequencesynthetic peptide
AKSPEKAKSPAEAKSP

SEQ ID NO: 42 - protein, 16 aa, Artificial Sequence
  Description of Artificial Sequencesynthetic peptide
AKSPEKAKSPAEAKSP

SEQ ID NO: 43 - protein, 16 aa, Artificial Sequence
  Description of Artificial Sequencesynthetic peptide
AKSPEKAKSPAEAKSP

SEQ ID NO: 44 - protein, 16 aa, Artificial Sequence
  Description of Artificial Sequencesynthetic peptide
AKSPEKAKSPAEAKSP

SEQ ID NO: 45 - protein, 16 aa, Artificial Sequence
  Description of Artificial Sequencesynthetic peptide
AKSPEKAKSPAEAKSP

SEQ ID NO: 46 - protein, 17 aa, Artificial Sequence
  Description of Artificial Sequencesynthetic peptide
AKSPEKAKSPAEAKSPA

SEQ ID NO: 47 - protein, 16 aa, Artificial Sequence
  Description of Artificial Sequencesynthetic peptide
AKSPEKAKSPEKAKSP

SEQ ID NO: 48 - protein, 16 aa, Artificial Sequence
  Description of Artificial Sequencesynthetic peptide
AKSPEKAKSPEKAKSP

SEQ ID NO: 49 - protein, 16 aa, Artificial Sequence
  Description of Artificial Sequencesynthetic peptide
AKSPEKAKSPEKAKSP

SEQ ID NO: 50 - protein, 16 aa, Artificial Sequence
  Description of Artificial Sequencesynthetic peptide
AKSPEKAKSPEKAKSP

SEQ ID NO: 51 - protein, 16 aa, Artificial Sequence
  Description of Artificial Sequencesynthetic peptide
AKSPEKAKSPEKAKSP

SEQ ID NO: 52 - protein, 16 aa, Artificial Sequence
  Description of Artificial Sequencesynthetic peptide
AKSPEKAKSPEKAKSP

SEQ ID NO: 53 - protein, 16 aa, Artificial Sequence
  Description of Artificial Sequencesynthetic peptide
AKSPEKAKSPEKAKSP

SEQ ID NO: 54 - protein, 17 aa, Artificial Sequence
  Description of Artificial Sequencesynthetic peptide
AKSPEKAKSPEKAKSPV

SEQ ID NO: 55 - protein, 18 aa, Artificial Sequence
  Description of Artificial Sequencesynthetic peptide
AKSPEKAKSPVKEEAKSP

SEQ ID NO: 56 - protein, 18 aa, Artificial Sequence
  Description of Artificial Sequencesynthetic peptide
AKSPEKAKSPVKEEAKSP

SEQ ID NO: 57 - protein, 18 aa, Artificial Sequence
  Description of Artificial Sequencesynthetic peptide
AKSPEKAKSPVKEEAKSP

SEQ ID NO: 58 - protein, 18 aa, Artificial Sequence
  Description of Artificial Sequencesynthetic peptide
AKSPEKAKSPVKEEAKSP

SEQ ID NO: 59 - protein, 18 aa, Artificial Sequence
  Description of Artificial Sequencesynthetic peptide
AKSPEKAKSPVKEEAKSP

SEQ ID NO: 60 - protein, 18 aa, Artificial Sequence
  Description of Artificial Sequencesynthetic peptide
AKSPEKAKSPVKEEAKSP

SEQ ID NO: 61 - protein, 18 aa, Artificial Sequence
  Description of Artificial Sequencesynthetic peptide
AKSPEKAKSPVKEEAKSP

SEQ ID NO: 62 - protein, 19 aa, Artificial Sequence
  Description of Artificial Sequencesynthetic peptide
AKSPEKAKSPVKEEAKSPV

SEQ ID NO: 63 - protein, 18 aa, Artificial Sequence
  Description of Artificial Sequencesynthetic peptide
AKSPVKEEAKSPAEAKSP

SEQ ID NO: 64 - protein, 18 aa, Artificial Sequence
  Description of Artificial Sequencesynthetic peptide
AKSPVKEEAKSPAEAKSP

SEQ ID NO: 65 - protein, 18 aa, Artificial Sequence
  Description of Artificial Sequencesynthetic peptide
AKSPVKEEAKSPAEAKSP

SEQ ID NO: 66 - protein, 18 aa, Artificial Sequence
  Description of Artificial Sequencesynthetic peptide
AKSPVKEEAKSPAEAKSP

SEQ ID NO: 67 - protein, 18 aa, Artificial Sequence
  Description of Artificial Sequencesynthetic peptide
AKSPVKEEAKSPAEAKSP

SEQ ID NO: 68 - protein, 18 aa, Artificial Sequence
  Description of Artificial Sequencesynthetic peptide
AKSPVKEEAKSPAEAKSP

SEQ ID NO: 69 - protein, 18 aa, Artificial Sequence
  Description of Artificial Sequencesynthetic peptide
AKSPVKEEAKSPAEAKSP

SEQ ID NO: 70 - protein, 19 aa, Artificial Sequence
  Description of Artificial Sequencesynthetic peptide
AKSPVKEEAKSPAEAKSPV

SEQ ID NO: 71 - protein, 18 aa, Artificial Sequence
  Description of Artificial Sequencesynthetic peptide
AKSPVKEEAKSPEKAKSP

SEQ ID NO: 72 - protein, 18 aa, Artificial Sequence
  Description of Artificial Sequencesynthetic peptide
AKSPVKEEAKSPEKAKSP

SEQ ID NO: 73 - protein, 18 aa, Artificial Sequence
  Description of Artificial Sequencesynthetic peptide
AKSPVKEEAKSPEKAKSP

SEQ ID NO: 74 - protein, 18 aa, Artificial Sequence
  Description of Artificial Sequencesynthetic peptide
AKSPVKEEAKSPEKAKSP

SEQ ID NO: 75 - protein, 18 aa, Artificial Sequence
  Description of Artificial Sequencesynthetic peptide
AKSPVKEEAKSPEKAKSP

SEQ ID NO: 76 - protein, 18 aa, Artificial Sequence
  Description of Artificial Sequencesynthetic peptide
AKSPVKEEAKSPEKAKSP

SEQ ID NO: 77 - protein, 18 aa, Artificial Sequence
  Description of Artificial Sequencesynthetic peptide
AKSPVKEEAKSPEKAKSP

SEQ ID NO: 78 - protein, 19 aa, Artificial Sequence
  Description of Artificial Sequencesynthetic peptide
AKSPVKEEAKSPEKAKSPV

SEQ ID NO: 79 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

(19) United States
(12) Patent Application Publication
(10) Pub. No.: US 2003/0148404 A1
(43) Pub. Date: Aug. 7, 2003

(54) Peptides and substances, methods and devices using same for diagnosing and treating neurodegenerative disorders

(75) Inventor: Daniel M. Michaelson, Tel Aviv (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(73) Assignee: Ramot University Authority For Applied Research & Industrial Development Ltd.

(21) Appl. No.: 09/847,586

(22) Filed: May 3, 2001

Related U.S. Application Data
(63) Continuation-in-part of application No. PCT/IL00/0059, filed on Aug. 27, 2000.
(60) Provisional application No. 60/221,150, filed on Jul. 27, 2000.

(51) Int. Cl. G01N 33/567; G01N 33/53; A61K 39/395

(52) U.S. Cl. 435/7.21; 435/7.5; 424/140.1

(57) ABSTRACT

A method of identifying an existence, non-existence, type or state of a neurodegenerative disorder in an individual. The method is effected by (a) immunoreacting with a serum sample derived from the individual at least one peptide representing at least one epitope derived from an endogenous protein to which at least one antibody is produced in vivo at onset or during progression of the neurodegenerative disorder, the at least one peptide being selected such that the at least one antibody being capable of immunobinding with the at least one peptide; and (b) detecting a presence, absence or degree of the immunobinding to thereby identify the existence, non-existence, type or state of the neurodegenerative disorder.

This application is a continuation in part of PCT application IL00/00509 filed Aug. 27, 2000, which claims the benefit of priority from U.S. patent application Ser. No. 09/386,347 filed Aug. 31, 1999.

### FIELD AND BACKGROUND OF THE INVENTION

The present invention relates to peptides derived from protein or proteins associated with a neurodegenerative disorder and to methods, substances and devices utilizing same. More particularly, the present invention relates to peptides representing immunogenic epitopes derived from a protein to which at least one antibody is produced in vivo at onset or during progression of a neurodegenerative disorder, such as, but not limited to, Alzheimer's disease. According to the teachings of the present invention the peptides can be used to (i) diagnose existence, non-existence, type or state of a neurodegenerative disorder; (ii) selectively remove an antibody from the blood of a patient suffering from the neurodegenerative disorder; and (iii) further characterize the neurodegenerative disorder. The present invention further relates to a method for identifying peptides useful for identifying an existence, non-existence, type or state of a neurodegenerative disorder in an individual. Citation or identification of any reference in this application shall not be construed as an admission that such reference is available as prior art to the present invention.

Alzheimer's Disease:

Alzheimer's Disease (AD) is a common form of neurodegenerative dementia of unknown cause. Alzheimer's Disease typically initiates in late middle age and characterized by progressive memory loss and mental deterioration, associated with brain damage, and resulting in relentlessly progressive intellectual and personality decline.

Brains of patients with AD contain characteristic extracellular senile plaques as well as intracellular neurofibrillary tangles [Katzman, 1976]. These histopathological changes are particularly pronounced in cortical and hippocampal areas and in the nuclei of the basal forebrain which provide most of the cholinergic input to the cortex and hippocampus [Coyle, 1983]. The severity of these degenerative changes correlates with the cognitive impairment in AD [Blessed, 1968], as well as with a reduction in central cholinergic activity [Francis, 1985]. The cholinergic changes are manifested by dysfunction and death of neurons in the basal forebrain and by a concomitant reduction in presynaptic cholinergic parameters in the cortex and the hippocampus [Sims, 1983]. The extent of the cholinergic deficit, its occurrence early in the disease, its correlation with the cognitive deficit in AD [Francis, 1985], and the known role of cholinergic mechanisms in higher cognitive functions, particularly memory [Bartus, 1985], all indicate a central role for cholinergic degeneration in the pathogenesis of AD.

Although AD is the commonest of the dementia, at present AD cannot be satisfactorily diagnosed during life and the quest for simple, non-invasive tests for diagnosis of AD is one of the highest priorities in the field.

Present Method for the Diagnosis of AD:

Today, the only accepted method to diagnose AD is neuropsychological testing, which enables analysis of a patient's cognitive skills, emotional, psychological, motor, and sensory attributes. Two examples of such tests are mini-mental status exam (MMSE) and the Blessed test [McDougall, 1990]. The former is a quick test, which is about 5 minutes long, that roughly assesses cognitive skills, reading, writing, orientation, and short-term memory. The latter test, in addition to the above mentioned faculties also evaluates activities associated with daily living. The accuracy of the neuropsychological tests is not very impressive [Forstl, 1998] and ranges between 70-90%, depending on the examiner. In view of the increasing need for accurate, fast and inexpensive diagnostics, spurned by the intense development of new drugs for the alleviation of symptoms and treatment of the disease, there is a real need for an impartial laboratory test.

Genetic Markers and Tests for AD:

Mutations in the genes presenilin-1 and presenilin-2 have been shown to lead to early onset familial AD [Perez-Tur, 1996; Perez-Tur, 1996; Perez-Tur, 1995; Crook, 1997]. The use of these markers is appropriate in cases when patients have a family history of the disease.

The association between the apolipoprotein E (apoE) gene and the risk to develop sporadic AD has been confirmed many times [Roses, 1998]. Studies have shown that there exists a linkage between the age of onset and the prevalence of AD and the apoE4 allele, which is a particular form of the apoE. However, many people carrying the apoE4 allele never develop AD and some AD patients do not posses the apoE4 allele. Therefore, this gene can not be used solely as a marker for AD but only as an additional confirmatory test in patients which are suspected of having AD. In such cases, the use of the apoE4 test reduced the false positive rate from 45% to 16%.

Variants of the A2M gene have been claimed by GenoPlex Inc. as a risk factor for sporadic AD [Blacker, 1998]. However, this claim has been refuted-by a study published in Nature Genetics [Rudrasingham, 1999; Dow, 1999; Wavrant-DeVrieze, 1999; Sherrington, 1995; Levy-Lahad, 1995]. No other genetic marker has been identified which can reliably predict the risk of sporadic AD. Furthermore, a true genetic marker for AD could not be used for tracking progression of the disease.

As such, the development of a reliable biochemical serological test is currently pursued by several research groups. Such a test must be based on the existence of a biochemical marker molecule, that ideally appears ahead of any symptoms (for early detection/screening) and which concentration in body fluids is proportional to the severity of the disease.

Biochemical Tests

Several molecules have been scrutinized as potential markers for predicting and/or diagnosing AD.

Amyloid B peptide and Tau in the cerebrospinal fluid (CSF) each used individually have been unreliable as AD markers. However, when used in combination, predictive reliability is increased and as such these markers are currently incorporated in a detection kit marketed by Athena Diagnostics. Nonetheless, the reliability of this marker combination is limited since the results obtained therewith suffer from excessive overlap between AD and non-AD patients, as well as situations where Tau and amyloid-protein levels are both either low or both high in which case determinations are not effective.

Neural thread protein (NTP) is marketed by Nymox Corporation as an early marker for AD [de la Monte, 1992; Monte, 1997]. It is present in neurons, is associated with neural plaques and is selectively upregulated in the AD brain [De La Monte, 1996]. The NTP diagnostic kit rely on detecting NTP in urine.

p97 is an iron-binding protein which has been shown to be present in the AD brain and to be specifically located in microglia cells in close association with senile plaques [Jefferies, 1996]. Studies have shown that significantly higher levels of p97 are present in AD sera as compared with normal control (NC) sera [Kennard, 1996]. Synapse Technologies Inc. is currently developing an AD marker system which utilizes this protein.

Immunological Mechanisms and AD:

Several reports indicate the involvement of immunological mechanisms in the etiology of AD. These include the presence of immunoglobulins (Igs) in senile plaques [Ishii, 1976; Eikelenboom, 1982], the presence of antibodies in AD sera which have been shown histochemically to react with neuronal tissue [Ishii, 1976; Eikelenboom, 1982; Nandy, 1978; Watts, 1981; Fillit, 1985], and abnormally increased expression of AHLA-DR antigens in brains of AD patients [Rogers, 1987; Pouplard-Barthelaix, 1987; McGeer, 1987]. Furthermore, the presence of immune complexes in the cerebrospinal fluid (CSF) of AD patients [Cameron, 1985] and defective cellular immune function have also been described [Skias, 1985; Singh, 1986].

AD Specific Antibodies

In view of the marked cholinergic degeneration in AD and of the suggested involvement of immunological mechanisms in the disease, a study was initiated to explore whether sera of AD patients contain antibodies that bind to specific constituents of cholinergic neurons [Chapman, 1988]. It was subsequently shown that AD sera contain a repertoire of antibodies directed against the heavy neurofilament subunit (NF—H), and that a subpopulation of these antibodies is specific to AD. It was also shown that this subpopulation of antibodies bind to NF—H epitopes the levels of which are significantly higher in neurofilaments of cholinergic neurons than in those obtained from heterogeneous neuronal preparations [Chapman, 1989; Soussan, 1994]. These epitopes were shown to be phosphorylated and located to the carboxyl terminal domain of the NF—H. Thus, the level and repertoire of anti-NF—H antibodies may reflect the extent and specificity of neuronal degeneration. Accordingly, neuronal degeneration, and increased leakage of the blood brain barrier leading to exposure of brain antigens to the immune system, that occurs either during normal aging or in AD may result in exposure and release of normal intracellular constituents, such as neurofilaments, and in the subsequent triggering of an immune response and of antibody production. Thus, since every class of neurons may exhibit NF—H with different epitopes, the specificity of the subclass of anti-NF—H antibodies present in the blood, may be used as a diagnostic tool not only for AD, but also for other neurodegenerative disorders that are brought about by the cell death of a distinct neuronal class. However, an alternative possibility should also be considered; AD is associated with aberrant phosphorylation of neurofilaments and of other cytoskeletal proteins [Sternberger, 1985; Grundke-Iqbal, 1986; Lichtenberg-Kraag, 1992; Masliah, 1993]. Also, NF—H from a purely cholinergic neuron preparation contains more than twofold more phosphorylated serine residues than does NF—H extracted from a heterogeneous neuron source [Soussan, 1994]. Thus, since the AD specific anti-cholinergic NF—H IgG bind to phosphorylated epitopes, it is possible that the specificity of these antibodies is caused by a cross reaction of antibodies that were generated in vivo against an abnormal antigen such as hyperphosphorylated neurofilaments or Tau.

There is thus a widely recognized need for, and it would be highly advantageous to have, a reliable method for the diagnosis and treatment of a neurodegenerative disorder, such as, for example Alzheimer's disease, from the early onset stage throughout the progression of the disease.

### SUMMARY OF THE INVENTION

According to the present invention there is provided a method of identifying an existence, non-existence, type or state of a neurodegenerative disorder in an individual, the method comprising the steps of (a) immunoreacting with a serum sample derived from the individual at least one peptide representing at least one epitope derived from an endogenous protein to which at least one antibody is produced in vivo at onset or during progression of the neurodegenerative disorder, the at least one peptide being selected such that the at least one antibody being capable of immunobinding with the at least one peptide; and (b) detecting a presence, absence or degree of the immunobinding to thereby identify the existence, non-existence, type or state of the neurodegenerative disorder.

According to another aspect of the present invention there is provided a proteinaceous substance useful for identifying an existence, non-existence, type or state of a neurodegenerative disorder in an individual, the proteinaceous substance comprising at least one peptide representing at least one epitope derived from an endogenous protein to which at least one antibody is produced in vivo at onset or during progression of the neurodegenerative disorder, the at least one peptide being selected such that the at least one antibody being capable of immunobinding the at least one peptide.

According to yet another aspect of the present invention there is provided a filter for removing at least one antibody generated against an endogenous protein associated with the onset or progression of the neurodegenerative disorder from the blood of a patient suffering from the neurodegenerative disorder, the filter comprising a solid support and the proteinaceous substance described hereinabove attached thereto such that filtering the blood of a patient suffering from the neurodegenerative disorder through the filter substantially removes the at least one antibody therefrom.

According to still another aspect of the present invention there is provided an extracorporeal device for removing at least one antibody generated against an endogenous protein associated with the onset or progression of a neurodegenerative disorder from the blood of a patient suffering from the neurodegenerative disorder, the extracorporeal device comprising (a) the filter described above; and (b) a pump for circulating the blood of the patient suffering from the neurodegenerative disorder through the filter, such that the at least one antibody is substantially removed from the blood of a patient.

According to yet an additional aspect of the present invention there is provided a peptide comprising an amino acid sequence representing at least one epitope of an endogenous protein to which at least one antibody is produced in vivo at onset or during progression of a neurodegenerative disorder.

According to still an additional aspect of the present invention there is provided a method of removing at least one antibody generated against an endogenous protein associated with the onset or progression of a neurodegenerative disorder from the blood of a patient suffering from the neurodegenerative disorder, the method comprising the step of circulating the blood of the patient through an extracorporeal device including at least one peptide representing at least one epitope derived from an endogenous protein and capable of immunobinding at least one antibody recognizing the endogenous protein and which is associated with the neurodegenerative disorder, the extracorporeal device is configured such that when the blood of the patient is circulated therethrough the at least one peptide immunobinds the at least one antibody to thereby substantially remove antibodies associated with the neurodegenerative disorder from the blood of the patient.

According to a further aspect of the present invention there is provided an array device useful for identifying an existence, non-existence, type or state of a neurodegenerative disorder in an individual, the array device comprising a plurality of peptides each being attached to a solid support in a regiospecific manner, the plurality of peptides representing epitopes derived from at least one endogenous protein to which a plurality of antibodies are produced in vivo at onset or during progression of the neurodegenerative disorder, each of the plurality of peptides being selected such that each of the plurality of antibodies being capable of immunobinding the at least each of the plurality of peptides.

According to a preferred embodiment of the invention described below, the endogenous protein is selected from the group consisting of NF—H, NF-M, Tau and B-amyloid protein.

According to still further features in the described preferred embodiments the at least one epitope is a continuous epitope.

According to still further features in the described preferred embodiments the at least one epitope a discontinuous epitope.

According to still further features in the described preferred embodiments the at least one peptide includes a number of amino acids selected from the group consisting of at least five, at least six, at least seven, at least eight, at least nine, at least ten, at least eleven, at least twelve,-at least thirteen, at least fourteen, at least fifteen, at least sixteen, at least seventeen, between seventeen and twenty five and between twenty five and at least thirty.

According to still further features in the described preferred embodiments the at least one peptide includes an amino acid sequence as set forth in SEQ ID NO:23.

According to still further features in the described preferred embodiments the at least one peptide includes a plurality of peptides and further wherein the at least one antibody includes a plurality of antibodies, whereas the plurality of peptides are selected such that the plurality of antibodies are capable of respectively immunobinding with the plurality of peptides.

According to still further features in the described preferred embodiments each of the plurality of peptides includes an amino acid sequence selected from the group consisting of SEQ ID NOs:5-76.

According to still further features in the described preferred embodiments each of the plurality of peptides is of an amino acid sequence selected from the group consisting of SEQ ID NOs: 21, 29, 32, 36, 38, 42, 44, 46, 54, 59, 62, 68, 70, 77 and 78.

According to still further features in the described preferred embodiments each of the plurality of peptides is of an amino acid sequence selected from the group consisting of SEQ ID NOs: 21, 32, 42, 54, 59, 62 and 77.

According to still further features in the described preferred embodiments the neurodegenerative disorder is associated with progressive loss of cognitive functions.

According to still further features in the described preferred embodiments the neurodegenerative disorder is associated with progressive loss of control of motoric functions.

According to still further features in the described preferred embodiments the neurodegenerative disorder is associated with progressive loss of motoric functions.

According to still further features in the described preferred embodiments the neurodegenerative disorder is selected from the group consisting of diseases accompanied by dementia, such as, but not limited to, Alzheimer's disease; Multi-infarct Dementia (MID); Pick's disease; Frontotemporal dementias with Parkinsonism linked to chromosome 17; Dementia pugilistica; Parkinson's disease with dementia; Gerstmann-Straussler-Scheinker disease with tangles; vascular dementia and neurodegenerative diseases not accompanied by dementia such as, but not limited to, Parkinson's disease; Multiple sclerosis; ALS; TIA and stroke without dementia.

According to still further features in the described preferred embodiments the at least one peptide includes an immobilizing moiety covalently attached thereto.

According to still further features in the described preferred embodiments the immobilizing moiety is a member of a binding pair.

According to still further features in the described preferred embodiments the member of the binding pair is selected from the group consisting of biotin, avidin, streptavidin, an antibody, a hapten, a receptor, a ligand, Ni and NTA.

According to still further features in the described preferred embodiments the immobilizing moiety is covalently attached to a terminal of the at least one peptide, the terminal is selected from the group consisting of a carboxy terminal and an amino terminal.

According to still further features in the described preferred embodiments at least one amino acid of the at least one peptide is a modified amino acid.

According to an additional aspect of the present invention there is provided a method of identifying peptides useful for identifying an existence, non-existence, type or state of a neurodegenerative disorder in an individual, the method comprising the steps of (a) preparing a plurality of peptides corresponding to a plurality of continuous or discontinuous sequences derived from an endogenous protein to which at least one antibody is produced in vivo at onset or during progression of the neurodegenerative disorder; (b) screening the plurality of peptides for at least one peptide being immunoreactive with a serum derived from at least one patient suffering from the neurodegenerative disorder, thereby identifying peptides useful of identifying an existence, non-existence, type or state of the neurodegenerative disorder According to still further features in the described preferred embodiments the continuous or discontinuous sequences derived from the endogenous protein include at least one phospho amino acid.

According to still further features in the described preferred embodiments the at least one phospho amino acid is selected from the group consisting of phosphoserine, phosphothreonine and phosphotyrosine.

According to still further features in the described preferred embodiments the phosphoserine forms a part of a sequence motif selected from the group consisting of sequence motives as set forth in SEQ ID NOs: 3, 4 and 5.

According to still further features in the described preferred embodiments the continuous or discontinuous sequences derived from the endogenous protein include at least one repeat of the sequence set forth by SEQ ID NO:2.

According to still further features in the described preferred embodiments the continuous or discontinuous sequences derived from the endogenous protein include at least one sequence motif selected from the group consisting of SEQ ID NOs: 1, 3 and 4.

According to still further features in the described preferred embodiments the step of preparing the plurality of peptides includes covalently attaching to each of the plurality of peptides at least one immoubilzing moiety.

According to still further features in the described preferred embodiments the immobilizing moiety is a member of a binding pair as further detailed above.

According to yet an additional aspect of the present invention there is provided a method of generating a peptide combination useful for identifying an existence, non-existence, type or state of a neurodegenerative disorder in an individual, the method comprising the steps of: (a) identifying at least one endogenous protein to which at least one antibody is produced in vivo at onset or during progression of the neurodegenerative disorder; (b) generating a plurality of peptides corresponding to the at least one endogenous protein; (c) reacting specific subsets of the plurality of peptide with serum obtained from: (i) a first population of individuals suffering from the neurodegenerative disorder; and (ii) a second population of individuals not suffering from the neurodegenerative disorder; and (d) identifying subset or subsets of the plurality of peptides being immunoreactive with a high number of said individuals of said first population and a low number of said individuals of said second population to thereby generate the peptide combination useful for identifying an existence, non-existence, type or state of a neurodegenerative disorder in an individual.

According to yet an additional aspect of the present invention there is provided a method of identifying an existence, non-existence, type or state of a neurodegenerative disorder in an individual, the method comprising the steps of: (a) immunoreacting a serum sample derived from the individual with a plurality of peptides, each peptide of the plurality of peptides representing at least one epitope derived from an endogenous protein to which at least one antibody is produced in vivo at onset or during progression of the neurodegenerative disorder; and (b) detecting a presence, absence or degree of antibody binding to each of the plurality of peptides to thereby generate an immunobinding profile for the serum sample derived from the individual, the profile being indicative of the existence, non-existence, type or state of the neurodegenerative disorder.

According to still further features in the described preferred embodiments the plurality of peptides are bound in a _regiospecific manner to a solid support, such that the immunobinding profile is generated by identifying reactive peptides of the plurality of peptides according to their regiospecificity.

According to still further features in the described preferred embodiments the plurality of peptides are overlapping peptides.

According to still further features in the described preferred embodiments each of the plurality of peptides includes a number of amino acids selected from the group consisting of at least five, at least six, at least seven, at least eight, at least nine, at least ten, at least eleven, at least twelve, at least thirteen, at least fourteen, at least fifteen, at least sixteen, at least seventeen, between seventeen and twenty five and between twenty five and at least thirty.

According to still further features in the described preferred embodiments the plurality of peptides are bound in a regiospecific manner to a solid support, such that reactive peptides are identifiable according to their regiospecificity.

According to still further features in the described preferred embodiments at least a portion of the plurality of peptides each include at least one phospho amino acid.

According to still further features in the described preferred embodiments the at least one phospho amino acid is selected from the group consisting of phosphoserine, phosphothreonine and phosphotyrosine.

The present invention successfully addresses the shortcomings of the presently known configurations by providing peptides and substances, and methods and devices utilizing these peptides and substances for diagnosing and treating neurodegenerative disorders.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 depicts the results obtained from blocked or unblocked plates assayed using a saturating peptide concentration. The plates were either blocked with 0.5% Gelatin, 1% Caseinate or not blocked, before the addition of the peptide. The serum was diluted in PBST and the secondary antibody in PBST containing either 0.5% Gelatin or 1% Caseinate.

FIG. 2 depicts detection results as a response to increasing concentrations of peptide, using either PBS or TBS as the reaction buffer.

FIG. 3 depicts detection results as a response to AD serum dilutions.

FIG. 4 depicts detection results as a response to secondary antibody-enzyme conjugate dilutions.

FIG. 5 represents the amino acid sequence of the Tau protein (SEQ ID NO:79). Regions within the protein which can be used to generate peptides according to the teachings of the present invention include boxed Serine and/or Threonine residue(s), at least one of which is phosphorylated.

FIG. 6 is a schematic depiction of an extracorporeal device for removing antibody or antibodies associated with a neurodegenerative disorder from the blood of a patient suffering from the disorder, according to the present invention.

FIG. 7 depicts an algorithm used to separate AD from normal control (NC) serum samples according to signals obtained using present invention.

FIG. 8 is schematic representation of profiles of antibody levels against different peptides, characteristic for AD or NC sera.

FIG. 9. illustrates various peptide combinations which enable distinction between four different pairs of test subject groups.

### DESCRIPTION OF THE PREFERRED EMBODIMENTS

The present invention is of a peptide or peptides which can be used to diagnose and/or treat a neurodegenerative disorder such as Alzheimer's Disease (AD). Specifically, the present invention can be used to detect a presence, or absence of an antibody or antibodies produced, in vivo, against an endogenous protein at onset or during progression of the neurodegenerative disorder, to thereby identify the existence, non-existence, type or state of the neurodegenerative disorder. The peptide or peptides according to the present invention can also be used to remove an antibody or antibodies produced, in vivo, against an endogenous protein, from the blood of a patient suffering from a neurodegenerative disorder, to thereby effect treatment of the neurodegenerative disorder. In addition, the present invention also provides a method of identifying peptides useful for identifying an existence, non-existence, type or state of a neurodegenerative disorder in an individual.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

As used herein, the term “treat” includes substantially inhibiting, slowing or reversing the progression of a disease, substantially ameliorating clinical symptoms of a disease or substantially preventing the appearance of clinical symptoms of a disease.

As used herein in the specification and in the claims section below the term “peptide” includes native peptides (either degradation products, synthetically synthesized peptides or recombinant peptides) and peptido-mimetics (typically, synthetically synthesized peptides), such as peptoids and semipeptoids which are peptide analogs, which may have, for example, modifications rendering the peptides more stable while in a body, or more immunogenic. Such modifications include, but are not limited to, cyclization, N terminus modification, C terminus modification, peptide bond modification, including, but not limited to, CH₂—NH, CH₂—S, CH₂—S═O, O═C—NH, CH₂—O, CH₂—CH₂, S═C—NH, CH═CH or CF═CH, backbone modification and residue modification. Methods for preparing peptido-mimetic compounds are well known in the art and are specified, for example, in Quantitative Drug Design, C. A. Ramsden-Gd., Chapter 17.2, F. Choplin Pergamon Press (1992), which is incorporated by reference as if fully set forth herein. Further detail in this respect are provided hereinunder.

Thus, a peptide according to the present invention can be a cyclic peptide. Cyclization can be obtained, for example, through amide bond formation, e.g., by incorporating Glu, Asp, Lys, Orn, di-amino butyric (Dab) acid, di-aminopropionic (Dap) acid at various positions in the chain (—CO—NH or —NH—CO bonds). Backbone to backbone cyclization can also be obtained through incorporation of modified amino acids of the formulas H—N((CH₂)ₙ—COOH)—C(R)H—COOH or H—N((CH₂)ₙ—COOH)—C(R)H—NH₂, wherein n=1-4, and further wherein R is any natural or non-natural side chain of an amino acid.

Cyclization via formation of S—S bonds through incorporation of two Cys residues is also possible. Additional side-chain to side chain cyclization can be obtained via formation of an interaction bond of the formula —(—CH₂—)ₙ—S—CH₂—C—, wherein n=1 or 2, which is possible, for example, through incorporation of Cys or homoCys and reaction of its free SH group with, e.g., bromoacetylated Lys, Orn, Dab or Dap.

Peptide bonds (—CO—NH—) within the peptide may be substituted, for example, by N-methylated bonds (—N(CH₃)—CO—), ester bonds (—C(R)H—C—O—O—C(R)—N—), ketomethylen bonds (—CO—CH₂—), α-aza bonds (—NH—N(R)—CO—), wherein R is any alkyl, e.g., methyl, carba bonds (—CH₂—NH—), hydroxyethylene bonds (—CH(OH)—CH₂—), thioamide bonds (—CS—NH—), olefinic double bonds (—CH═CH—), retro amide bonds (—NH—CO—), peptide derivatives (—N(R)—CH₂—CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as TIC, naphthylelanine (Nol), ring-methylated derivatives of Phe, halogenated derivatives of Phe or o-methyl-Tyr.

Tables 1-2 below list all the naturally occurring amino acids (Table 1) and non-conventional or modified amino acids (Table 2).

TABLE 1

| | Three-Letter | |
|---|---|---|
| Amino Acid | Abbreviation | One-letter Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Iie | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE 2

| Non-conventional amino acid | Code | Non-conventional amino acid | Code |
|---|---|---|---|
| α-aminobutylic acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methyhmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl) glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomo | Mhphe |
| | | phenylalanine | |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α-methylvaline | Mtrp | L-α-methyltyrosine | Mtyr |
| L-α-methylleucine | Mval | L-N-methylhomophenylalanine | Nmhphe |
| | Nnbhm | | |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1-(2,2-diphenyl | Nmbc | | |
| ethylamino)cyclopropane | | | |

A peptide according to the present invention can be used in a self standing form or be a part of moieties such as proteins and display moieties such as display bacteria and phages.

Additionally, a peptide according to the present invention includes at least five, optionally at least six, optionally at least seven, optionally at least eight, optionally at least nine, optionally at least ten, optionally at least eleven, optionally at least twelve, optionally at least thirteen, optionally at least fourteen, optionally at least fifteen, optionally at least sixteen or optionally at least seventeen, optionally between seventeen and twenty five or optionally between twenty five and at least thirty amino acid residues (also referred to herein interchangeably as amino acids).

Accordingly, as used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

As used herein in the specification and in the claims section below the phrase “derived from a protein” refers to peptides derived from the specified protein or proteins and further to homologous peptides derived from equivalent regions of proteins homologous to the specified proteins of the same or other species, provided that these peptides are effective for the detection of antibodies associated with a neurodegenerative disorder. The term further relates to permissible amino acid alterations and peptido-mimetics designed based on the amino acid sequence of the specified proteins or their homologous proteins.

As used herein the term “epitope” and the phrase “antigenic determinant” both refer to a region of a molecule, such as, for example, the peptide(s) of the present invention, which region is characterized by specific molecular arrangement so as to be recognized and bound by a specific antibody species. When derived from a molecule which is linear by nature, yet acquires a complex three dimensional structure in which regions which are distant from one another in the linear topography are close to one another in the complex three dimensional structure, such as a protein, an epitope can either be continuous, i.e., defined by a contiguous sequence, or discontinuous, i.e., defined by a combination of at lest two non-contiguous regions of the sequence.

As used herein the term “antibody” also refers to “antibody species” or “monospecific antibody” and is used to define an antibody subset which is of the same clonal origin and which therefore reacts with a single epitope. Antibodies of any Ig class can be targeted by the peptides of the present invention, of preferable targeting are presently antibodies of the IgG and IgM classes which are present in the blood serum.

As used herein the phrase “self antibody” refers to antibodies produced against epitopes which form a part of a self (endogenous) protein. The production of self antibodies in an individual often results in what is known as an “autoimmune response”.

As used herein the phrase “antibody(s) associated with a neurodegenerative disorder” refers to antibody or antibodies which are directed against an endogenous protein, which antibodies are produced in vivo at onset or during the progression of a neurodegenerative disorder.

As used herein the phrase “neurodegenerative disorder” is used to define a disorder characterized by progressive loss of cognitive functions, progressive loss of control of motoric functions and/or progressive loss of motoric functions. Such disorders can include diseases accompanied by dementia, such as, but not limited to, Alzheimer's disease; Multi-infarct Dementia (MID); Pick's disease; Frontotemporal dementias with Parkinsonism linked to chromosome 17; Dementia pugilistica; Parkinson's disease with dementia; Gerstmann-Straussler-Scheinker disease with tangles; vascular dementia and neurodegenerative diseases not accompanied by dementia such as, but not limited to, Parkinson's disease; Multiple sclerosis; ALS; TIA and stroke without dementia.

As already mentioned hereinabove, Alzheimer's Disease (AD) is a common form of neurodegenerative dementia of unknown cause.

AD is partially characterized by the presence, in cholinergic neurons, of a variant of the heavy neurofilament subunit (NF—H), which variant contains a significantly higher level of hyperphosphorylated epitopes than NF—H found in heterogeneous neuronal cells. It has been shown that AD sera contain a repertoire of antibodies directed against these epitopes of NF—H, and that a subpopulation of these antibodies is specific to AD. It has further been shown that a large portion of this antibody subpopulation is specific to the carboxy terminal of this protein.

Based on this information it was hypothesized that it might be possible to associate certain antibody species found in individuals with the onset or progression of AD or other neurodegenerative disorders.

In order to test this hypothesis, and while reducing the present invention to practice, a set of peptides which represent the epitopes of the carboxy terminal of NF—H were generated and screened against sera of AD and non-AD individuals. Candidate peptides were identified, which can be used for diagnosing AD.

Since NF—H has a linear configuration, the carboxy domain thereof can be represented with overlapping peptides that span the entire molecule. Although this is in general a sound approach, it would require hundreds of peptides to represent the whole length in all potential phosphorylation states.

The peptide approach becomes manageable due to the characteristic sequence and organization of the carboxy terminal domain. This domain is composed of numerous repeats of only three sequences. Each of these sequences is 6 to 8 amino acids long and it contains an AKSP (SEQ ID NO:2) motif, the serine of which when contained within the native NF—H molecule represents a potential phosphorylation site [Soppet, 1992]. Thus, the specific configuration of the NF—H molecule allows to construct a small number of phosphorylated and non-phosphorylated peptides which span the entire length of the relevant NF—H domain. As is further detailed in the Examples section that follows, peptides generated according to the teachings of the present invention have been utilized with great success in specifically identifying sera of AD patients. It will be appreciated that the same peptide design logic that was applied to NF—H can be applied to other proteins associated with neurodegenerative disorders in which the formation of self antibodies is observed. Such protein candidates can include, but are not limited to, NF-M, Tau (either in solution or as insoluble tangle), B-amyloid protein or peptides derived from B-amyloid protein (in solution or in the form of insoluble plaques).

Thus, according to the teachings of the present invention, there is provided a method of identifying peptides useful for identifying an existence, non-existence, type or state of a neurodegenerative disorder in an individual.

The method according to this aspect of the present invention is implemented by executing the following method steps, in which, in a first step, a plurality of peptides corresponding to a plurality of continuous or discontinuous sequences derived from an endogenous protein, to which at least one antibody is produced in vivo at onset or during progression of the neurodegenerative disorder, are prepared. Preferably the endogenous sequence is first computer analyzed for theoretical antigenic determinants, by for example, the software provided by the Genetic Computer Group package of the Wisconsin University (GCG). Such computer characterization of possible antigenic determinants provides further information useful for peptide planning.

In a second step of the method according to this aspect of the present invention, the plurality of peptides are screened for the presence of at least one peptide which is differentially immunoreactive (e.g., not immunoreactive or which is substantially less or more immunoreactive) with a serum derived from a normal control individual, as is compared to a serum derived from a patient suffering from the neurodegenerative disorder.

Peptides thus identified can then be used for identifying an existence, non-existence, type or state of a neurodegenerative disorder. This can be effected, for example, by correlating a specific set or sets of immunoreactive peptides (profile) with the existence, non-existence, type or state of a specific neurodegenerative disorder.

It will be appreciated that several screening approaches can be used in context with this aspect of the present invention, which approaches include, but are not limited to, enzyme linked immuno-sorbent assay (ELISA), immunopercipitation, western blots, slot and dot blots, magnetic bead separation, solid support arrays, affinity columns and phage or bacterial display. These methods are well known in the art and as such no further description thereof is provided herein.

It will be appreciated in this case that when a peptide or peptides are used in context with screening methods which include a solid or semisolid support, the peptide(s) preferably include a binding moiety such that the peptide can be immobilized to such supports.

Thus according to another preferred embodiment of the present invention, the peptide(s) further include an immobilizing moiety covalently attached thereto. Such an immobilizing moiety can be a charged moiety which can electrostatically bind surface charges provided on the support. Alternatively and preferably, the immobilizing moiety is a member of a binding pair which can bind to its co-member when the latter is attached to the solid support. Examples of such binding pairs include, but are not limited to, biotin-avidin/streptavidin, antibody-antigen/hapten, e.g., a peptide tag such as FLAG c-myc and the like, cellulose binding domain (CBD)-cellulose, receptor-ligand and Ni-NTA. One member of the binding pair can be covalently bound to the peptide, for example, at the amino or carboxy terminal, and the other member of the binding pair can be covalently or otherwise bound to the support, such that the immobilization of the peptide on the support is provided by the interaction between the members of the binding pair. Alternatively, peptides can be attached directly to a solid support by reacting an amino- or carboxyl group of the peptide with a reactive group which forms a part of the solid support.

Peptides according to the teachings of the present invention can be synthesized by standard peptide synthesis techniques, for example, using either standard 9-fluorenylmethoxycarbonyl (F-Moc) chemistry [see, for example, Atherton, 1985] or standard butyloxycarbonate (T-Boc) chemistry although it is noted that, more recently, the fluorenylmethoxycarbonyl (Fmoc)/tert-butyl system, developed by Sheppard et al. has found increasingly wide application [Sheppard, 1986]. The correctness of the structure and the level of purity, which will normally be in excess of 85%, should be carefully checked, and particular attention be given to the correctness of internal disulfide bridging arrangements when present. Various chromatographic analyses, including high performance liquid chromatography (HPLC), and spectrographic analyses, including Raman spectroscopy, may, for example, be employed for this purpose. It will be appreciated that any suitable synthesis method may also be employed to synthesize peptide(s) directly on a solid support. Methods for synthesizing peptides on solid supports are well known in the art [for further detail see Bodanszky, 1985; Coe, 1998; Sucholeiki, 1998; Albericio, 1997] Using any of these methods, an immobilizing moiety or any other moiety or modified amino acid can readily be incorporated into a synthesized peptide.

It is to be understood that the peptides according to the present invention may be synthesized by any conventional method, either directly using manual or automated peptide synthesis techniques as mentioned above, or indirectly by RNA or DNA synthesis and conventional techniques of molecular biology and genetic engineering. Such techniques may be used to produce hybrid proteins containing one or more of the polypeptides fused into another polypeptide sequence such as the case of the bacterial or phage display mentioned above in context with screening methods.

It should be noted however that incorporating modified amino acids cannot be made directly using a recombinant DNA system. As such, since some of the peptides of the present invention include modifications such as phosphorylation, these peptides are preferably chemically synthesized as described hereinabove. It will be appreciated however that since directed phosphorylation can be provided by some cell expression system, such peptides can also be produced by recombinant techniques, although in this case, verification of phosphorylation should be employed prior to use.

Once peptides which are specifically reactive with serum of an individual suffering from a neurodegenerative disorder have been identified using the screening method of the present invention as hereinabove described, such peptides can be used to implement additional aspects of the present invention as further detailed in the following sections.

Thus, according to another aspect of the present invention, there is provided at least one peptide, preferably a set of peptides, which are utilizable for diagnosing or treating a neurodegenerative disorder, such as Alzheimer's disease. The utilization of such peptide or peptides for the diagnosis and/or treatment of a neurodegenerative disorder is further described hereinbelow.

The peptides according to the present invention each include an amino acid sequence representing at least one continuous or discontinuous epitope derived from an endogenous protein to which at least one antibody is produced in vivo at onset or during progression of a neurodegenerative disorder. This endogenous protein is defined as a protein normally expressed in the body of an individual and which, the over expression, specific localization and/or modification thereof is associated with a neurodegenerative disorder.

According to preferred embodiments of this aspect of the present invention the at least one epitope derived from the endogenous protein is either a continuous epitope or a discontinuous epitope. As is well known in the art of immunology, epitopes present in peptides and proteins are defined by the residues of the amino acid within the sequence of the peptide or protein and/or the modifications, such as the addition of prosthetic groups, to these amino acids. In any case, an epitope is determined by either a continuous or discontinuous stretch of amino acids which typically includes at least five to seven amino acids. It will be appreciated that an epitope is determined by either the primary structure (the sequence of amino acids) and/or by the spatial conformation of this sequence which can be determined by the secondary, tertiary, globular (quaternary) structure, or any combinations thereof.

According to a preferred embodiment of the present invention peptides derived from the endogenous protein each represent at least one epitope of this protein, such that antibodies reactive with the protein are also reactive with these peptides. It will be appreciated that peptides encompassing all the possible epitopes, continuous or discontinuous, which are included within an endogenous protein can be generated, according to the teachings of the present invention. For reasons further detailed hereinunder, generating all or a substantial fraction of such epitopes is preferably effected by phage or bacterial display, whereas generating a smaller fraction can be efficiently effected by peptide libraries, as is further exemplified in the Examples section that follows in context of the NF—H and Tau proteins.

According to another preferred embodiment of the present invention the peptide(s) include at least one phospho-amino acid. According to still another preferred embodiment of the present invention the phospho-amino acid is phosphoserine. It will be appreciated however, that other phosphorylated amino acids can be used in context with the peptides of the present invention, especially phosphorylated forms of amino acids which have been demonstrated to be associated with motifs found in proteins associated in one way or another with neurodegenerative disorders. Such phosphorylated amino acids include, but are not limited to, phosphotyrosine and phosphothreonine.

According to another preferred embodiment of the present invention, the phosphoserine forms a part of a sequence motif AKSP as set forth in SEQ ID NO:2. Alternatively, the phosphoserine forms a part of a sequence motif as set forth in SEQ ID NOs: 3, 4 or 5, each of which includes an AKSP core.

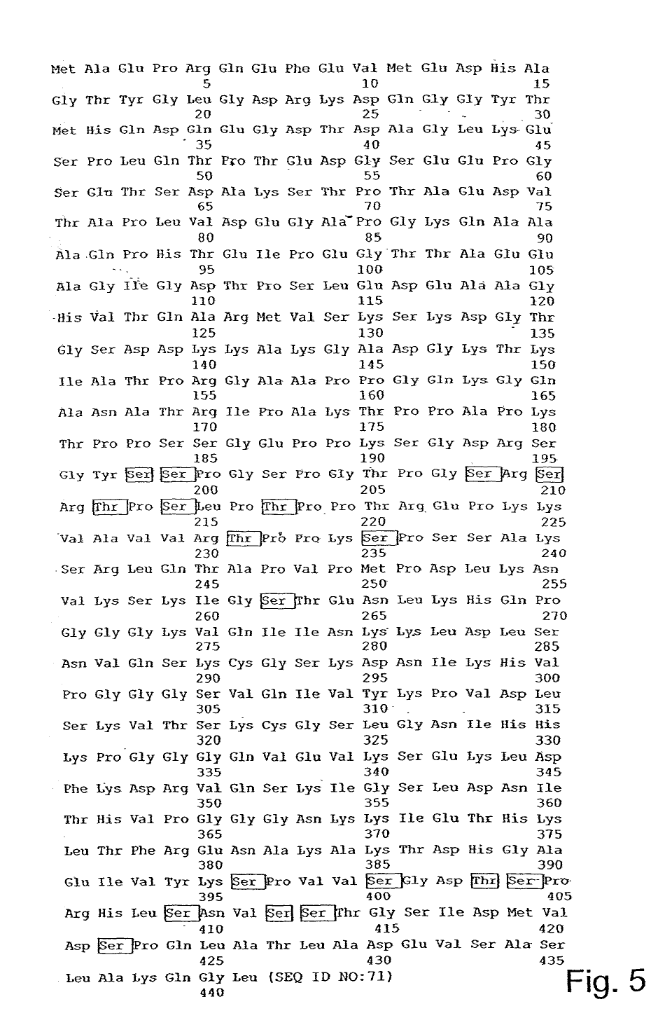

According to a preferred embodiment of the present invention, the endogenous protein is Tau, antibodies to which characterize AD patients. FIG. 5 represents the amino acid sequence of Tau (SEQ ID NO:79). Preferred regions within the protein which can be used to generate peptides according to the teachings of the present invention include boxed serine and threonine residues, at least one of which is phosphorylated. According to another preferred embodiment of the present invention, the endogenous protein is NF—H, antibodies to which characterize AD patients.

SEQ ID NOs:5-76 represent peptides generated according to the teachings of the present invention and which represent epitopes derived from the carboxy terminal of NF—H. Preferably a subset including, for example, some of the peptides set forth in SEQ ID NOs:5-76 is utilized for the detection of antibodies associated with AD. More preferably, a subset including some of the peptides set forth in SEQ ID NOs: 21, 29, 32, 36, 38, 42, 44, 46, 54, 59, 62, 68, 70, 77 and 78 or most preferably a subset including the peptides set forth in SEQ ID NOs: 21, 32, 42, 54, 59, 62 and 77 are utilized for the detection of antibodies associated with AD.

It will be appreciated that using the above described method, of identifying peptides useful for identifying an existence, non-existence, type or state of a neurodegenerative disorder in an individual, additional peptides derived from endogenous proteins associated with neurodegenerative disorders can be similarly synthesized and characterized.

Techniques and approaches for isolating and characterizing proteins associated with disorders which involve the generation of self antibodies in an individual are well known in the art. As such, applying these techniques and approaches to the field of neurodegenerative disorders one ordinarily skilled in the art, could design an approach suitable for isolating proteins against which antibodies are generated at onset or progression of a neurodegenerative disorder. For example, expression libraries, preferably subtraction expression libraries of sequences characterizing affected organs or regions thereof can be manufactured and screened against patient vs. normal control derived antibodies to thereby uncover new proteins against which antibodies are generated at onset or during the progression of a neurodegenerative disorder.

The sequence of such novel disease associated proteins can be utilized to generate short peptides of 5-25 amino acids in length spanning the novel protein. Single peptides or subsets of peptides can then be tested against serum derived from a population of individuals suffering from a neurodegenerative disorder and serum derived from a healthy population to thereby uncover peptides or subset of peptides which are most accurate in predicting the disease state.

Thus, the present invention is also applicable to yet uncovered proteins against which antibodies are generated at onset or during the progression of a neurodegenerative disorder.

As already mentioned hereinabove, one or more peptides according to the present invention can be presented in context of non-related amino acid sequences.

Thus, according to still another aspect of the present invention there is provided a proteinaceous substance useful for identifying an existence, non-existence, type or state of a neurodegenerative disorder in an individual which includes at least one peptide representing at least one epitope derived from an endogenous protein to which at least one antibody is produced in vivo at onset or during progression of the neurodegenerative disorder.

According to the present invention the proteinaceous substance is preferably immobilized. Such immobilization is preferably effected as described hereinabove, with respect to immobilizing moieties. Alternatively, immobilization can be effected by translationally fusing the peptide DNA sequence to a carrier DNA which codes for a carrier protein. This carrier protein-peptide fusion protein, when expressed by specific display systems enables displaying the peptide on the exterior portion of a bacteria or phage. Methods of constructing display libraries are well known in the art, such methods are described, for example, in Young AC, et al., “The three-dimensional structures of a polysaccharide binding antibody to Cryptococcus neoformans and its complex with a peptide from a phage display library: implications for the identification of peptide mimotopes” J Mol Biol Dec. 12, 1997; 274(4):622-34; Giebel L B et al. “Screening of cyclic peptide phage libraries identifies ligands that bind streptavidin with high affinities” Biochemistry Nov. 28, 1995; 34(47):15430-5; Davies E L et al., “Selection of specific phage-display antibodies using libraries derived from chicken immunoglobulin genes” J Immunol Methods Oct. 12, 1995; 186(1):125-35; Jones C et al. “Current trends in molecular recognition and bioseparation” J Chromatogr A Jul. 14, 1995; 707(1):3-22; Deng S J et al. “Basis for selection of improved carbohydrate-binding single-chain antibodies from synthetic gene libraries” Proc Natl Acad Sci USA May 23, 1995; 92(11):4992-6; and Deng S J et al. “Selection of antibody single-chain variable fragments with improved carbohydrate binding by phage display” J Biol Chem Apr. 1, 1994; 269(13):9533-8, which are incorporated herein by reference.

One main advantage of using display libraries, as opposed to peptide libraries, is the ability to dramatically increase the repertoire of sequences displayed because such sequences need not be presented in a regiospecific context as is the case for peptide libraries which are not propagatable.

Display libraries according to this aspect of the present invention can be used to detect binding to antibodies associated with a neurodegenerative disorder. As a result, screening for suitable peptides and identification of the existence, non-existence, type or state of the neurodegenerative disorder can be effected. Positive isolates, either phages or bacteria, can be thereafter directly employed in the diagnosis of patients in a fashion similar to that described above for self standing peptides.

Thus, as detailed hereinabove and in the Examples section which follows, according to the present invention peptides suitable for the specific immunobinding of antibodies which are produced in vivo at onset or during progression of a neurodegenerative disorder, such as Alzheimer's disease (AD), Multiple Infarct Dementia (MID) and Parkinson's Disease with Dementia (PwD) are provided. In addition, the present invention also provides an approach which can be used to identify new peptides derived from characterized and in the future from yet to be characterized endogenous proteins which are associated with self antibody production in neurodegenerative disorders.

As further detailed hereinunder, peptides synthesizable according to the present invention can be utilized as tools with which the identification and treatment of individuals possessing a neurodegenerative disorder can be effected. In addition, these peptides can also be used to further characterize neurodegenerative disorders.

Thus, according to yet another aspect of the present invention, there is provided a method for identifying an existence, non-existence, type or state of a neurodegenerative disorder in an individual.

The method according to this aspect of the present invention is effected by implementing the following method steps, in which, in a first step, a serum sample derived from the individual is immunoreacted with peptide(s) which are prepared according to the present invention. The reaction can be effected through several approaches some of which are listed hereinabove.

As used herein the term “serum” refers to mammalian blood or any portion or derivative thereof, treated or untreated. Preferably it refers to a blood sample from which hematopoietic cells have been removed.

According to a preferred embodiment of the present invention, the serum sample is reacted with a plurality of peptides which are arrayed on a solid support, as further detailed hereinunder. An example to possible reaction conditions and components is given in the Examples section that follows. It will be appreciated in this case, however, that other reaction parameters and components which enable the detection of a reaction can be employed by a skilled artisan while implementing the present invention.

In a second step of the method according to this aspect of the present invention, the detection of a presence, absence or the degree of immunobinding between the at least one peptide and an antibody contained within the serum sample is effected (profile). This enables to identify the existence, non-existence, type or state of the neurodegenerative disorder. The detection of binding can be visualized, for example, calorimetrically, fluoroscentically or be otherwise realized by any other method commonly practiced in the art, such as, for example, radioactivity counting and the like. It will be appreciated that these methods can be employed either manually or automatically. For example, it is possible, and it is further exemplified in the Examples section that follows, to use ELISA detection along with automated sample processing to yield detection. Alternatively, technologies for automated detection of microarrayed samples can be employed. Many examples of microarray detection systems exist in the art. The use of peptide loaded microchips is envisaged.

As already mentioned hereinabove, and according to another aspect of the present invention, peptides synthesizable according to the teachings of the present invention are preferably utilized in an array configuration for identifying an existence, non-existence, type or state of a neurodegenerative disorder in an individual. In the array configuration each of the plurality of peptides is attached to a solid support in a regiospecific manner to form an array device. This enables the recognition of positively reacted peptides according to their regiospecific location or attachment to the solid support.

Each of the plurality of peptides can represent a single epitope, or alternatively a plurality of epitopes derived from an endogenous protein to which a plurality of antibodies are produced in vivo at onset or during the progression of a neurodegenerative disorder. Thus, a positive immunobinding reaction detected for each of the peptides utilized can be indicative of the existence, non-existence, type or state of a neurodegenerative disorder. It will be appreciated that the use of an array device allows for the co-analysis of multiple immunobinding reactions and characterization of disorder specific profiles useful in precise identification of the existence, non-existence, type or state of the disorder. Furthermore, in cases where a state, type or existence of a neurodegenerative disorder is represented by a specific subset of antibodies, which are reactive to several endogenous proteins, co-analysis of multiple immunobinding reactions can enable the more precise identification of a specific state, type or existence of the disorder. This is particularly advantageous in cases where treatment of certain neurodegenerative disorders is most effective when specifically designed according to the state of progression or type of the disorder. As such, the recognition of a specific state or type of a neurodegenerative disorder can potentially enable a more effective treatment thereof. Employing a combination of peptides as herein described enables the detection of neurodegenerative disorders in general. Specific sets which includes various combinations of peptides would enable detection of different neurodegenerative disorders and type and progression states thereof.

Once practiced on a wide scale and in a universal fashion, correlations between progression states or types of a plurality of neurodegenerative disorders and patterns of arrayed immunoreactive peptides (profiles) will be established to thereby facilitate in automated diagnosis.

Since neurodegenerative disorders such as AD typically result from a complex syndrome rather than a singular cellular event, the fact that the peptides employed can measure a variety of antibody species, and not a single biochemical marker, broadens the range of detectable subtypes of neurodegenerative disorders, and as a result, increases the general sensitivity of the diagnosing method provided by the present invention. In sharp distinction, prior art methods and kits measure single biochemical markers, and as a result, such prior art methods typically and inherently enable the detection of a single subtype of a single neurodegenerative disorder.

As already mentioned hereinabove, peptides synthesizable according to the teachings of the present invention can be used for treating a neurodegenerative disorder by removing antibody(s) associated with the neurodegenerative disorder from the blood of a patient suffering from the disorder.

Thus, according to still another aspect of the present invention, there is provided a filter for removing antibody(s) associated with a neurodegenerative disorder from the blood of a patient suffering from the neurodegenerative disorder. The filter includes a solid support and an attached proteinaceous substance which includes a peptide or peptides according to the present invention. The filter can include a single type of peptide or alternatively it can include several types representing several epitopes associated with a single or several endogenous protein associated with a single neurodegenerative disorder. The proteinaceous substance is further described hereinabove. Thus to remove antibody(s) associated with a neurodegenerative disorder, the blood of a patient is circulated through the filter, such that the peptide(s) contained therein bind the antibody(s) associated with the neurodegenerative disorder contained within the blood.

It will be appreciated that the term “filter” is used herein to refer to any element which is capable of supporting the attached proteinaceous substance while at the same time allow for the blood of a patient to flow through in a manner which enables intimate contact between the blood components and the peptide(s) included within the proteinaceous substance. As such it is meant to include columns, membranes and the like.

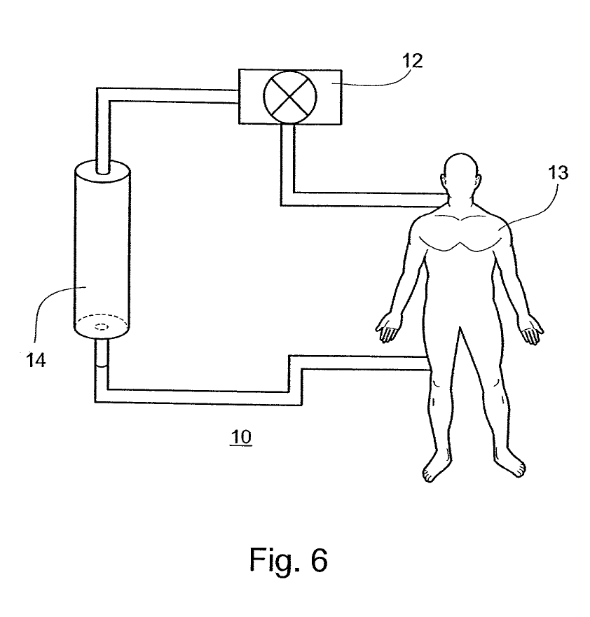

According to another aspect of the present invention there is provided an extracorporeal device designed or adapted for removing antibody(s) associated with a neurodegenerative disorder from the blood of a patient suffering from the neurodegenerative disorder. An example to such an extracorporeal device is shown in FIG. 6, and is referred to hereinbelow as device 10.

Device 10 includes a pump 12 for circulating the blood of a patient 13 suffering from the neurodegenerative disorder through a filter 14, which includes peptide or peptides as previously described herein above. By circulating the blood of a patient through device 10, antibody(s) associated with the neurodegenerative disorder are substantially removed from the blood of patient 13. It will be appreciated that in cases where these antibodies generate an autoimmune response which contributes to the onset or progression of the disorder, the removal of these antibodies would greatly diminish, or abolish the progression of the disorder.

It will be appreciated that many examples of blood filtering devices are known in the art included in which are dialysis machines and the like. As such, these devices can be readily modified into device 10 of the present invention.

Thus, the present invention provides peptides with which a neurodegenerative disorder can be diagnosed and treated. Furthermore, the present invention provides a method with which new peptides of characterized and yet to be characterized endogenous proteins associated with self antibody production in neurodegenerative disorders can be identified. In addition, the present invention provides devices for diagnosing and treating neurodegenerative disorders, which devices incorporate the peptide(s) according to the present invention. Finally, since the peptides of the present invention represent epitopes of proteins which are associated with neurodegenerative disorders, such peptides can also be used to further investigate and characterized such disorders.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

### Rational

Structure and characteristics of NF antigens: Since it was shown that a subset of NF—H associated antibodies is present at higher levels in AD than in negative control subjects [Chapman, 1988; Chapman, 1989], one may deduce that the NF—H molecule can be used to detect these antibodies present in blood serum. To do so, one must first characterize the structure of the molecule.

As taught by Soussan (1996), neurofilaments, a major constituent of the neuronal cytoskeleton, are composed of three different proteins. These subunits are called the heavy (NF—H), the medium (NF-M) and the light (NF-L) proteins, and their approximate molecular masses are 200, 160, and 68 kDa, respectively. All the neurofilament proteins contain a conserved helical rod domain which forms the basis of their polymerization and assembly to 10 nm wide filaments. The remaining carboxy terminal domains of the neurofilament proteins, particularly those of the larger subunits NF—H and NF-M, form side arms which extend from the helical core of the neurofilament fiber and cross-bridge it to adjacent neurofilaments or to other cytoskeletal elements [Robinson, 1988; Steinert, 1988]. These extended carboxy terminal tail domains contain multiple repeats of the sequence motif Lys-Ser-Pro (KSP, SEQ ID NO:1) which repeat approximately 10 times in NF-M and more than 40 times in NF—H. The serine residues in these repeating KSP sequences are heavily phosphorylated and serve as substrates for second messenger-independent kinases [Julien, 1983; Lee, 1988; Wible, 1989; Roder, 1991]. Neurofilament proteins can also be phosphorylated by second messenger-dependent kinases including protein kinase C, cyclic AMP-dependent protein kinase, and Ca²⁺/calmodulin-dependent kinase [Gonda, 1990; Sihag, 1990; Tokui, 1990; Dosemeci, 1992]. The sites phosphorylated by the latter kinases, however, are situated in the amino terminal end of the neurofilament subunits and are much less abundant than those of the repeating KSP motif [Nixon, 1991]. The extent of phosphorylation of neurofilament proteins is developmentally controlled and varies between different parts of the neuron [Dahl, 1983; Sternberger, 1983; Lee, 1987; Dahl, 1988; Carden, 1987]. This evidence, some of which is further discussed hereinabove, is also provided from immunohistochemical and immunoblot experiments which have shown that specific monoclonal antibodies directed against phosphorylated and non-phosphorylated neurofilament epitopes yield distinct binding patterns in different types of neurons [Campbell, 1989; Szaro, 1990; Vickers, 1990; Berglund, 1991; Clark, 1991; Faigon, 1991].

Synthetic peptide approach to the detection of AD specific antibodies: NF—H has been successfully used as an antigen in antibody capture assays, where it was shown that AD-sera contain markedly and significantly higher levels of anti-NF—H antibody as compared with normal control (NC) sera [Chapman, 1988; Chapman, 1989]. Moreover, when the native NF—H molecule was replaced with the highly phosphorylated carboxy terminal tail of the NF—H as the antigen in the immunoassay, the separation between signals obtained from AD and NC sera was further improved [Soussan, 1994].

It is not practical to use the whole NF—H molecule or its carboxy domain-for a commercial in vitro diagnostic kit for AD because it would be prohibitively expensive to produce large amounts of this big post-translationally modified protein. However, since this molecule has a linear configuration, while conceiving the present invention it was hypothesized that one could conceptually represent the carboxy domain with overlapping synthetic peptides that span the entire molecule.

Although this is in general a sound approach, it would require hundreds of peptides to represent the whole length in all potential phosphorylation states.

A synthetic peptide approach becomes manageable in this case due to the characteristic sequence and organization of the carboxy terminal domain. This domain is composed of numerous repeats of only three sequences. Each of these sequences is 6 to 8 amino acids long and it contains an AKSP (SEQ ID NO:2) motif, the serine of which when contained within the native NF—H molecule represents a potential phosphorylation site [Soppet, 1992]. Thus, the specific configuration of the NF—H molecule allows to construct a small number of phosphorylated and non-phosphorylated peptides which span the entire length of the relevant NF—H domain. These peptides can then be used for a systematic “epitope walk” along the molecule. The validity and potential of the NF—H “epitope walk” approach are further strengthened by the fact that NF—H has an extended linear conformation and that many of the NF—H antigenic sites which are recognized by the AD antibodies are resistant to denaturation. For further details the reader is referred to [Nixson, 1991].

Design of synthetic neurofilament peptides: An epitope is determined by a stretch of up to 7 to 8 amino acids. Thus, in order to mimic the antigenic properties of the tail domain of NF—H with synthetic peptides, it is necessary to calculate the total number of possible amino acid sequences which contain potential phosphorylation sites (e.g., AKSP, SEQ ID NO:2) which are flanked by 3 to 8 amino acids on each side. The sequence of the NF—H tail domain is composed of the following three repeating amino acid motifs:

| | | |
|---|---|---|
| (i) | A K S P A; | (motif A, SEQ ID NO:3) |
| (ii) | A K S P E K; and | (motif B, SEQ ID NO:4) |
| (iii) | A K S P V K E E | (motif C, SEQ ID NO:5) |

Considering the possible arrangements of these motifs along the NF—H molecules [Soppet, 1992] and the fact that an epitope can be determined by up to seven to eight amino acids, the entire length of the NF—H tail domain can be represented by the following eight peptides:

| | | |
|---|---|---|
| (1) | AKSPAEAKSPAEAKSP; | (SEQ ID NO:6) |
| (2) | AKSPAEAKSPEKAKSP; | (SEQ ID NO:7) |
| (3) | AKSPAEAKSPVKEEAKSP; | (SEQ ID NO:8) |
| (4) | AKSPEKAKSPAEAKSP; | (SEQ ID NO:9) |
| (5) | AKSPEKAKSPEKAKSP; | (SEQ ID NO:10) |
| (6) | AKSPEKAKSPVKEEAKSP; | (SEQ ID NO:11) |
| (7) | 0AKSPVKEEAKSPAEAKSP; and | (SEQ ID NO:12) |
| (8) | AKSPVKEEAKSPEKAKSP | (SEQ ID NO:13) |

Each of these peptides has a potential serine phosphorylation site in the middle, which is part of a KSP, and which is flanked by two additional KSP (SEQ ID NO:1) moieties. Thus, each peptide can exist in eight different states of phosphorylation. Accordingly a total of 64 such peptides covers all the possible states of phosphorylation of the NF—H carboxy terminal domain, because motif C (SEQ ID NO:5) does not occur in tandem in the naturally occurring protein.

The peptide sequences selected are as follows (all peptides are biotinylated at the N-terminal):

| | | |
|---|---|---|
| (1NO) | A K S P A E A K S P A E A K S P-OH | (SEQ ID NO:6) |
| (1L) | A K S(PO₃H) P A E A K S P A E A K S P-OH | (SEQ ID NO:14) |
| (1M) | A K S P A E A K S(PO₃H) P A E A K S P-OH | (SEQ ID NO:15) |
| (1R) | A K S P A E A K S P A E A K S(PO₃H) P-OH | (SEQ ID NO:16) |
| (1LM) | A K S(PO₃H) P A E A K S(PO₃H) P A E A K S P-OH | (SEQ ID NO:17) |
| (1LR) | A K S(PO₃H) P A E A K S P A E A K S(PO₃H) P-OH | (SEQ ID NO:18) |
| (1MR) | A K S P A E A K S(PO₃H) P A E A K S(PO₃H) P-OH | (SEQ ID NO:19) |
| (1LMR) | A K S(PO₃H) P A E A K S(PO₃H) P A E A K S(PO₃H) P-OH | (SEQ ID NO:20) |
| (1LMR-A) | A K S(PO₃H) P A E A K S(PO₃H) P A E A K S(PO₃H) P A-NH₂ | (SEQ ID NO:21) |
| (2NO) | A K S P A E A K S P E K A K S P-OH | (SEQ ID NO:7) |
| (2L) | A K S(PO₃H) P A E A K S P E K A K S P-OH | (SEQ ID NO:22) |
| (2M) | A K S P A E A K S(PO₃H) P E K A K S P-OH | (SEQ ID NO:23) |
| (2R) | A K S P A E A K S P E K A K S(PO₃H) P-OH | (SEQ ID NO:24) |
| (2LM) | A K S(PO₃H) P A E A K S(PO₃H) P E K A K S P-OH | (SEQ ID NO:25) |
| (2LR) | A K S(PO₃H) P A E A K S P E K A K S(PO₃H) P-OH | (SEQ ID NO:26) |
| (2MR) | A K S P A E A K S(PO₃H) P E K A K S(PO₃H) P-OH | (SEQ ID NO:27) |
| (2LMR) | A K S(PO₃H) P A E A K S(PO₃H) P E K A K S(PO₃H) P-OH | (SEQ ID NO:28) |
| (2LMR-A) | A K S(PO₃H) P A E A K S(PO₃H) P E K A K S(PO₃H) P A-NH₂ | (SEQ ID NO:29) |
| (3NO) | A K S P A E A K S P V K E E A K S P-OH | (SEQ ID NO:8) |
| (3L) | A K S(PO₃H) P A E A K S P V K E E A K S P-OH | (SEQ ID NO:30) |
| (3M) | A K S P A E A K S(PO₃H) P V K E E A K S P-OH | (SEQ ID NO:31) |
| (3R) | A K S P A E A K S P V K E E A K S(PO₃H) P-OH | (SEQ ID NO:32) |
| (3LM) | A K S(PO₃H) P A E A K S(PO₃H) P V K E E A K S P-OH | (SEQ ID NO:33) |
| (3LR) | A K S(PO₃H) P A E A K S P V K E E A K S(PO₃H) P-OH | (SEQ ID NO:34) |
| (3MR) | A K S P A E A K S(PO₃H) P V K E E A K S(PO₃H) P-OH | (SEQ ID NO:35) |
| (3MR-V) | A K S P A E A K S(PO₃H) P V K E E A K S(PO₃H) P V-NH₂ | (SEQ ID NO:36) |
| (3LMR) | A K S(PO₃H) P A E A K S(PO₃H) P V K E E A K S(PO₃H) P-OH | (SEQ ID NO:37) |
| (3LMR-V) | A K S(PO₃H) P A E A K S(PO₃H) P V K E E A K S(PO₃H) P V-NH₂ | (SEQ ID NO:38) |
| (4NO) | A K S P E K A K S P A E A K S P-OH | (SEQ ID NO:9) |
| (4L) | A K S(PO₃H) P E K A K S P A E A K S P-OH | (SEQ ID NO:39) |
| (4M) | A K S P E K A K S(PO₃H) P A E A K S P-OH | (SEQ ID NO:40) |
| (4R) | A K S P E K A K S P A E A K S(PO₃H) P-OH | (SEQ ID NO:41) |
| (4LM) | A K S(PO₃H) P E K A K S(PO₃H) P A E A K S P-OH | (SEQ ID NO:42) |
| (4LR) | A K S(PO₃H) P E K A K S P A E A K S(PO₃H) P-OH | (SEQ ID NO:43) |
| (4MR) | A K S P E K A K S(PO₃H) P A E A K S(PO₃H) P-OH | (SEQ ID NO:44) |
| (4LMR) | A K S(PO₃H) P E K A K S(PO₃H) P A E A K S(PO₃H) P-OH | (SEQ ID NO:45) |
| 4LMR-A) | A K S(PO₃H) P E K A K S(PO₃H) P A E A K S(PO₃H) P A-NH₂ | (SEQ ID NO:46) |
| (5NO) | A K S P E K A K S P E K A K S P-OH | (SEQ ID NO:10) |
| (5L) | A K S(PO₃H) P E K A K S P E K A K S P-OH | (SEQ ID NO:47) |
| (5M) | A K S P E K A K S(PO₃H) P E K A K S P-OH | (SEQ ID NO:48) |
| (5R) | A K S P E K A K S P E K A K S(PO₃H) P-OH | (SEQ ID NO:49) |
| (5LM) | A K S(PO₃H) P E K A K S(PO₃H) P E K A K S P-OH | (SEQ ID NO:50) |
| (5LR) | A K S(PO₃H) P E K A K S P E K A K S(PO₃H) P-OH | (SEQ ID NO:51) |
| (5MR) | A K S P E K A K S(PO₃H) P E K A K S(PO₃H) P-OH | (SEQ ID NO:52) |
| (5LMR) | A K S(PO₃H) P E K A K S(PO₃H) P E K A K S(PO₃H) P-OH | (SEQ ID NO:53) |
| (5LMR-V) | A K S(PO₃H) P E K A K S(PO₃H) P E K A K S(PO₃H) P V-NH₂ | (SEQ ID NO:54) |
| (6NO) | A K S P E K A K S P V K E E A K S P-OH | (SEQ ID NO:11) |
| (6L) | A K S(PO₃H) P E K A K S P V K E E A K S P-OH | (SEQ ID NO:55) |
| (6M) | A K S P E K A K S(PO₃H) P V K E E A K S P-OH | (SEQ ID NO:56) |
| (6R) | A K S P E K A K S P V K E E A K S(PO₃H) P-OH | (SEQ ID NO:57) |
| (6LM) | A K S(PO₃H) P E K A K S(PO₃H) P V K E E A K S P-OH | (SEQ ID NO:58) |
| (6LR) | A K S(PO₃H) P E K A K S P V K E E A K S(PO₃H) P-OH | (SEQ ID NO:59) |
| (6MR) | A K S P E K A K S(PO₃H) P V K E E A K S(PO₃H) P-OH | (SEQ ID NO:60) |
| (6LMR) | A K S(PO₃H) P E K A K S(PO₃H) P V K E E A K S(PO₃H) P-OH | (SEQ ID NO:61) |
| (6LMR-V) | A K S(PO₃H) P E K A K S(PO₃H) P V K E E A K S(PO₃H) P V-NH₂ | (SEQ ID NO:62) |
| (7NO) | A K S P V K E E A K S P A E A K S P-OH | (SEQ ID NO:12) |
| (7L) | A K S(PO₃H) P V K E E A K S P A E A K S P-OH | (SEQ ID NO:63) |
| (7M) | A K S P V K E E A K S(PO₃H) P A E A K S P-OH | (SEQ ID NO:64) |
| (7R) | A K S P V K E E A K S P A E A K S(PO₃H) P-OH | (SEQ ID NO:65) |
| (7LM) | A K S(PO₃H) P V K E E A K S(PO₃H) P A E A K S P-OH | (SEQ ID NO:66) |
| (7LR) | A K S(PO₃H) P V K E E A K S P A E A K S(PO₃H) P-OH | (SEQ ID NO:67) |
| (7MR) | A K S P V K E E A K S(PO₃H) P A E A K S(PO₃H) P-OH | (SEQ ID NO:68) |
| (7LMR) | A K S(PO₃H) P V K E E A K S(PO₃H) P A E A K S(PO₃H) P-OH | (SEQ ID NO:69) |
| (7LMR-V) | A K S(PO₃H) P V K E E A K S(PO₃H) P A E A K S(PO₃H) P V-NH₂ | (SEQ ID NO:70) |
| (8NO) | A K S P V K E E A K S P E K A K S P-OH | (SEQ ID NO:13) |
| (8L) | A K S(PO₃H) P V K E E A K S P E K A K S P-OH | (SEQ ID NO:71) |
| (8M) | A K S P V K E E A K S(PO₃H) P E K A K S P-OH | (SEQ ID NO:72) |
| (8R) | A K S P V K E E A K S P E K A K S(PO₃H) P-OH | (SEQ ID NO:73) |
| (8LM) | A K S(PO₃H) P V K E E A K S(PO₃H) P E K A K S P-OH | (SEQ ID NO:74) |
| (8LR) | A K S(PO₃H) P V K E E A K S P E K A K S(PO₃H) P-OH | (SEQ ID NO:75) |
| (8MR) | A K S P V K E E A K S(PO₃H) P E K A K S(PO₃H) P-OH | (SEQ ID NO:76) |
| (8LMR) | A K S(PO₃H) P V K E E A K S(PO₃H) P E K A K S(PO₃H) P-OH | (SEQ ID NO:77) |
| (8LMR-V) | A K S(PO₃H) P V K E E A K S(PO₃H) P E K A K S(PO₃H) P V-NH2 | (SEQ ID NO:78) |

Tau—an alternative protein candidate: The Tau protein, which physiologically stabilizes microtubules in the neuronal axon, is an integral constituent of paired helical filaments (PHF), which form neurofibrillary tangles. Hyperphosphorylation of Tau has been considered the main cause of PHF assembly [Goedert, 1992] although alternatively, this protein could be involved in a secondary event in PHF formation.

Using the same paradigm for Tau as described herein for NF—H, and considering the fact that Tau, like NF—H, is a linear protein which is hyperphosphorylated in AD, it is possible to dissect the sequence of Tau and design a number of peptides that represents the entire hyperphosphorylated region, in its different phosphorylation states. These peptides can be used as potential tools for the detection of anti-Tau antibodies which are specific to AD patients. FIG. 5 presents the amino acid sequence of Tau. Peptides of 6-30 amino acid residues containing at least one phosphorylated serine or threonine among the boxed serines and threonines can serve as peptides for implementing the present invention.

### Example 2

### Materials and Experimental Methods

Binding Peptides to a Solid Support:

Introduction to enzyme linked immunosorbent assay (ELISA): ELISA is a convenient method for measuring concentration of antigens or antibodies in solution. In principle, the substance to be measured is bound to a solid phase and then specifically detected by an enzyme-labeled antibody. The enzyme generates a color reaction, the optical density (OD) of which is proportional to its concentration. Thus, with excess reagents the OD is proportional to the amount of substance bound to the solid phase. To measure antibody concentration in serum it is common to bind an antigen thereof to the solid support. As there are countless numbers of antigens and many kind of solid supports, the variations are endless. However, the most commonly used solid support is polystyrene in the form of a plate with 96 microwells arranged as an 8 by 12 array. The polystyrene can be treated to modify the electrostatic and hydrophobic binding forces of the plastic surface. There are plates commercially available that are optimized for maximum or minimum binding of a variety of ligands. Large proteins often bind readily to polystyrene. Thus, to measure the concentration of a certain antibody, its antigen, the protein, is nonspecifically adsorbed to the surface of the microwell. The antibody-containing solution is then added and after binding of the antibody to the protein, the non-specific antibodies are washed out. In the next step, an anti-antibody antibody labeled with an enzyme which catalyzes a color reaction is added and the complex and detection is effected by adding the chromogenic substrate to the enzyme.

When the ligand to be used for capturing the antibody is small, for example, a short peptide, the non-covalent binding forces between the short peptide and the plastic surface are usually too weak to prevent the short peptide from being washed out. If the peptide is negatively charged at working pH, this problem can be traversed by precoating the wells with poly-L-lysine which is positively charged, and as such binds the peptide to the plate with electrostatic bonds. It is also common practice to add the peptide-solution to the well, evaporate all the liquid and immobilize the peptide to the surface by fixation with Methanol. Alternatively, plates made of plastic containing reactive groups that specifically bind amino- or carboxyl groups, enabling covalent binding of the peptides to the plate surface, can be used. However, these plates are useful only for peptides which contain only a single amino- or carboxyl group. Although peptides rich in Lysine such as the peptides employed herein, can be used in conjunction with such plates, such use is not preferred since these peptides can bind to the plate by means of the internal Lysine residues, and not necessarily through the terminal group. This prevents the binding of the peptide in a specific configuration, causes a large fraction of the peptides to bind parallel to the surface and as a consequence renders these peptides inaccessible to the antibodies in the serum.

All the above mentioned methods were attempted in context of the present invention yet produced unreliable results. The OD obtained from wells to which a peptide had been added, was not significantly higher than the OD values of control wells to which the peptide has not been added.

Bead-based detection of antibodies: An alternative strategy was to use peptides conjugated to micron sized beads. In this assay the serum is added to the beads in a reaction tube. At the end of the incubation period, the beads are collected (spun down) and the >supernatant removed. The beads are washed and detection is performed. This system worked far better than the plate-based methods used previously. However, this method is extremely cumbersome and time consuming, variations between identical samples are quite big, and reproducibility between experiments can be problematic.

Streptavidin-biotin based method: The method that proved most reliable and successful is based on peptides biotinylated at the amino-terminal end and streptavidin coated multi-well plates. As further detailed hereinunder, this method gave very good signal-to-noise ratio, it proved to be very sensitive in a wide range of serum concentrations, and provided good reproducibility between samples, plates and repeated experiments.

Solutions and Materials: Tris Buffered Saline (TBS): 50 mM Tris-HCl, pH 7.4 (Tris (Sigma, T-1378); HCl (Merck 1.00319.1000)) 200 mM NaCl (Merck, 1.06404.1000). TBST: 0.05% (w/v) Tween-20 (Sigma, P-7949) in TBS. Phosphate Buffered Saline (PBS): 0.1 M phosphate buffer, pH 7.2 (Sodium phosphate, monobasic (Sigma. S-8182) and Sodium phosphate, dibasic (Sigma, S-7907)). 200 mM NaCl (Merck, 1.06404.1000). PBST: 0.05% (w/v) Tween-20 (Sigma, P-7949) in PBS. Dilution/Blocking buffer: 0.5% Gelatin (Difco, 0143-17-9) in TBST. Streptavidin stock solution: 2 mg/ml Streptavidin (Sigma, S4762) in purified water. Aliquoted and kept at −20° C., this solution is stable indefinitely. Multiwell Plates: Nunc Maxisorp (Cat. No. 442404) 96 well C-shaped microplates. Secondary antibody: Goat anti-human IgG Horse radish peroxidase conjugate (Jackson, 109-035-088). Substrate solution: 1 mg/ml OPD (Sigma, P-8412)+0.005% H₂O₂ (Merck, 1.07210.0250) in 50 nM Sodium Citrate buffer, pH 5 (Merck, 1.00 244.1000).

A Streptavidin stock solution (2 mg/ml Streptavidin prepared in purified water, aliquoted, and stored at −20° C.) was diluted 1:400 in ddH₂O and a 100 μl aliquot was added to each well of a multiwell plate (96 well C-shaped microplates). The plates were incubated at 37° C. overnight until the liquid was entirely evaporated and stored until use at 4° C. in plastic bags containing a desiccating material. Such coated plates retained their activity for at least few days. The plates were washed 4 times by immersing in TBST. 200 μl of blocking solution were added to each of the wells. The plates were then incubated for 1 hour at room temperature. 100 μl of the biotinylated peptide (1 μg/ml in TBST) were added to each of the wells with the exception of the control wells. The plates were then incubated for 1 hour at room temperature.

Following incubation, the plates were washed in TBST 4 times as above. A 100 μl aliquot of serum which was diluted in a dilution buffer was added to each well and the plates were incubated for 1 hour at room temperature.

Following this incubation the wells were washed in TBST 4 times as above. A 100 μl aliquot of a secondary antibody (Goat anti-human IgG Horse radish peroxidase conjugate) which was diluted 1:2000 in dilution buffer was added to each well and the plates were incubated for 1 hour at room temperature.

The wells were then washed in TBST 4 times as above followed by two washes in TBS (TBST w/o Tween-20).

A 100 μl aliquot of the substrate solution was added to each well and the plates were incubated at room temperature.

OD was measured at 450 nm and 620 nm every minute over a period of 10-15 minutes.

Peptide combinatorics: AD is a highly heterogenic neurodegenerative disorder, and it is therefor unlikely that one single biomarker will be able to detect all cases. Furthermore, preliminary results suggest that some peptides detect antibodies in the blood of certain cases, while other peptides are more efficient for the detection of antibodies of other cases. By employing a set of peptides, this apparent characteristic of a single peptide can be turned into an advantage. For example, if a certain serum sample analyzed with a given set of peptides produces a combined signal larger than an empirically set cut-off value, this serum sample is considered positive. Furthermore, the relative proportions attributed to individual peptides, when used in combination, do not have to be proportional to the signal, as such, freedom in the optimization of the inclusion criteria can be obtained.

### EXAMPLE 3

### Experimental Results

Optimization of the Protocol:

The peptide used for optimization was peptide 3M (SEQ ID NO:31). Serum was pooled from 10 AD patients and pretreated with chloroform.

To maximize binding while minimizing the background the following conditions were optimized: (i) blocking of non-specific binding of IgG; (ii) concentration of the peptide; (iii) buffer-system; (iv) serum concentration range; (v) serum incubation time and temperature; (vi) concentration of the secondary antibody; and (vii) pretreatment of the sera to remove lipids.

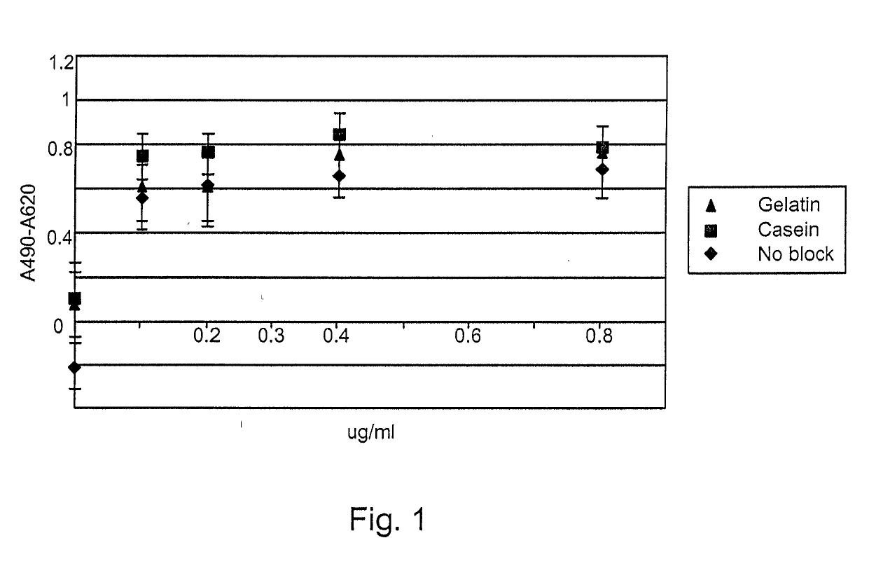

Blocking: The plates were either blocked with 0.5% Gelatin, 1% Caseinate or not blocked, before the addition of peptide. The serum was diluted in PBST and the secondary antibody in PBST containing either 0.5% Gelatin or 1% Caseinate. As seen in FIG. 1, a saturating concentration of the peptide was reached at 0.4-0.8 μg/ml. Accordingly, 1 μg/ml was chosen as a standard working concentration. The different blocking agents used did not substantially effect the signal, but improved reproducibility (see Table 1 hereinbelow). As such, 0.5% Gelatin was chosen as the blocking agent for subsequent experiments.

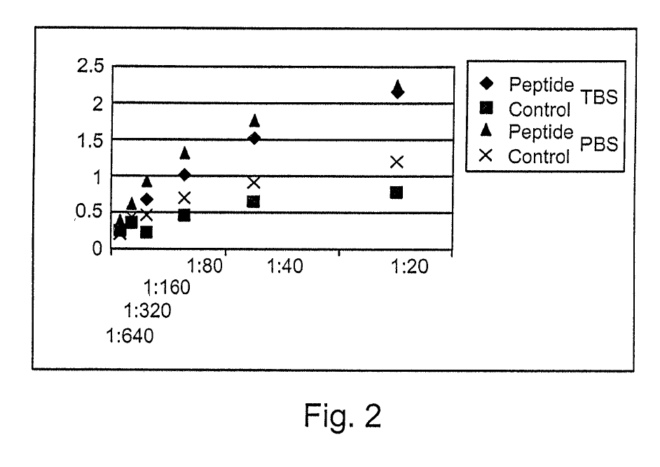

Buffers: The original protocol was based on phosphate buffered solutions (PBS). Since the specificity of IgG binding to the peptide is dependent on the phosphorylation state of the epitopes, presence of phosphate in the solution may, or may not, interfere with the assay. As such, Tris buffered solutions (TBS) were preferably employed. FIG. 2, depicts the response to increasing concentrations of the peptide, employed in either PBS or TBS. It is evident from this Figure that TBS functions at least as well if not better than PBS. Therefore, TBS was used as the preferred buffer for all subsequent experiments.

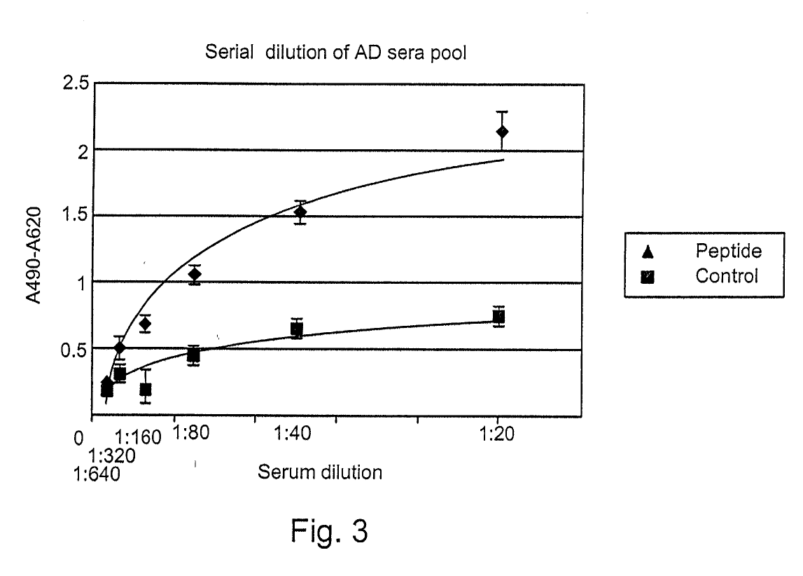

Serum concentration range: As seen in FIG. 3, detection is most sensitive to changes in serum concentration between 1:80 and 1:320, although detection can also be seen at concentrations between 1:20 and 1:40. Signal to noise ratios were maximal (˜3) at 1:20 and decreased below 2 at 1:160 (FIG. 2). When eight AD and eight normal control (NC) samples were analyzed at different dilutions (1:10-1: 640), it was clear that concentrations below 1:40 gave poor separation between AD and NC, while using concentration higher than 1:40 did significantly improve the detection (not shown). Based on these results, a working dilution of 1:40 was used for all subsequent experiments. Serum incubation time and temperature: Three different conditions for serum incubation were tested on eight AD and eight NC samples. In addition to the original protocol's one hour at room temperature, over-night incubations at room temperature or 4° C. were tested. The longer incubation times did not significantly improve signals from weakly positive samples, the same samples were generally identified as positive in all three conditions employed (not shown). Based on these results, the serum was incubated for one hour at room temperature in all subsequent experiments.

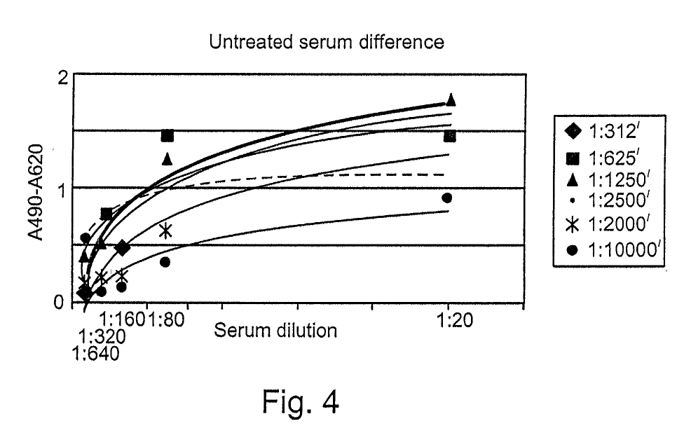

Concentration of the secondary antibody: Five different serum dilutions were probed with six different secondary antibody-enzyme conjugate concentrations. As seen in FIG. 4, the highest sensitivity was reached when dilutions 1:1250 and 1:2500 were employed. As such, a working dilution of 1:2000 was employed in all subsequent experiments.

Pretreatment of the sera for the removal lipids: Preliminary experiments included a lipid removal step. This step employed the addition of chloroform (5% chloroform, v/v) to the sera followed by gentle agitation for 30 minutes at room temperature, and 30 minutes of centrifugation at 20,000×g at 4° C. The sera (supernatant) was then separated from the chloroform-lipid pellet. However, in subsequent experiments it was shown that no significant difference were observed between signals obtained from the “treated” or “untreated” sera (not shown). As a result all subsequent experimentations were preferably conducted without employing this lipid removal step.

Preliminary Comparison Between AD and NC Sera:

Employing the above optimized parameters, eight AD and eight NC sera were analyzed using peptide 3M (SEQ ID NO:31). The experiment was repeated over a period of four days, employing four different plates each day. Two plates were preblocked with Gelatin, while two were not. In one experiment chloroform treated serum was analyzed on two additional plates. Three AD sera consistently gave rise to high signals in all experiments. One AD serum caused high signals in 10 out of the 13 plates and four AD sera were consistently negative. One NC sera caused high signals in some of the plates in the experiments conducted on all four days. Two additional NC sera gave rise to high signals in some of the plates in the experiments conducted on three of the four days. It was observed that when plates were coated with Streptavidin one day prior to the experiment, in contrast to the plates coated in advance and stored at 4° C. for 1-2 weeks, produced much better inter-plate reproducibility (not shown). The reproducibility of the experiments was assessed by averaging the rank-order numbers of each sample in every experiment (Table 3).

TABLE 3
Averaged rank-order numbers for 16 AD and NC samples

| Not preblocked | | | Preblocked | | |
|---|---|---|---|---|---|
| Sample No. | Average | STD | Sample No. | Average | STD |
| 862* | 1.17 | 0.29 | 862* | 1.17 | 0.29 |
| 871* | 2.33 | 0.76 | 871* | 2.67 | 1.26 |
| 861* | 2.83 | 1.04 | 861* | 3.33 | 1.04 |
| 874* | 6.67 | 2.02 | 874* | 4.83 | 1.04 |
| 485 | 7.67 | 4.25 | 485 | 5.83 | 1.44 |
| 465 | 8.17 | 2.02 | 465 | 7.50 | 4.50 |
| 895 | 9.00 | 2.50 | 899 | 9.33 | 2.52 |
| 869* | 9.33 | 4.80 | 869* | 10.00 | 3.77 |
| 910 | 9.83 | 1.76 | 873* | 10.00 | 1.73 |
| 913 | 10.17 | 1.89 | 895 | 10.17 | 5.53 |
| 909 | 10.33 | 5.25 | 870* | 10.50 | 2.78 |
| 870* | 10.50 | 1.80 | 910 | 10.67 | 1.26 |
| 479 | 10.83 | 0.58 | 909 | 10.83 | 2.31 |
| 899 | 11.33 | 2.47 | 872* | 11.83 | 3.55 |
| 872* | 12.83 | 2.47 | 479 | 13.67 | 0.29 |
| 873* | 13.00 | 2.18 | 913 | 13.67 | 2.25 |

Three different experiments performed on different days, with each experiment conducted on pre-blocked or non-blocked identical plates.
*AD sera; STD - standard deviation

As seen in Table 3, using pre-blocked plates, as opposed to non-blocked plates resulted in a larger statistical variation between the samples. Sample numbers 862, 871, 861, and 874, acquired from AD patients, are separated by more than two standard deviation points from sample number 465. Sample number 485 is separated by one standard deviation point from sample number 465. As such, peptide 3M detected four out of eight AD cases while generating one false positive signal (sample No. 485). It will be appreciated that these results are based on the employment of a single peptide. However, by combining several peptides both the specificity and sensitivity can be improved.

In the non-blocked samples, only three samples gave consistently high signals. It was therefore concluded that pre-blocking of the plates increases the reproducibility of the assay.

It will be appreciated that the protocol optimization was done with one single peptide. It was shown that the system is sensitive to changes in peptide concentration, serum concentration and secondary antibody concentration, as a result, optimal conditions were chosen. A preliminary study on a limited number of AD and NC samples gave a sensitivity of 50% (4/8) and specificity of 87% (⅞). Using a combination of several peptides will increase both the sensitivity and accuracy. As is shown below, using a combination of several peptides increases both the sensitivity and accuracy.

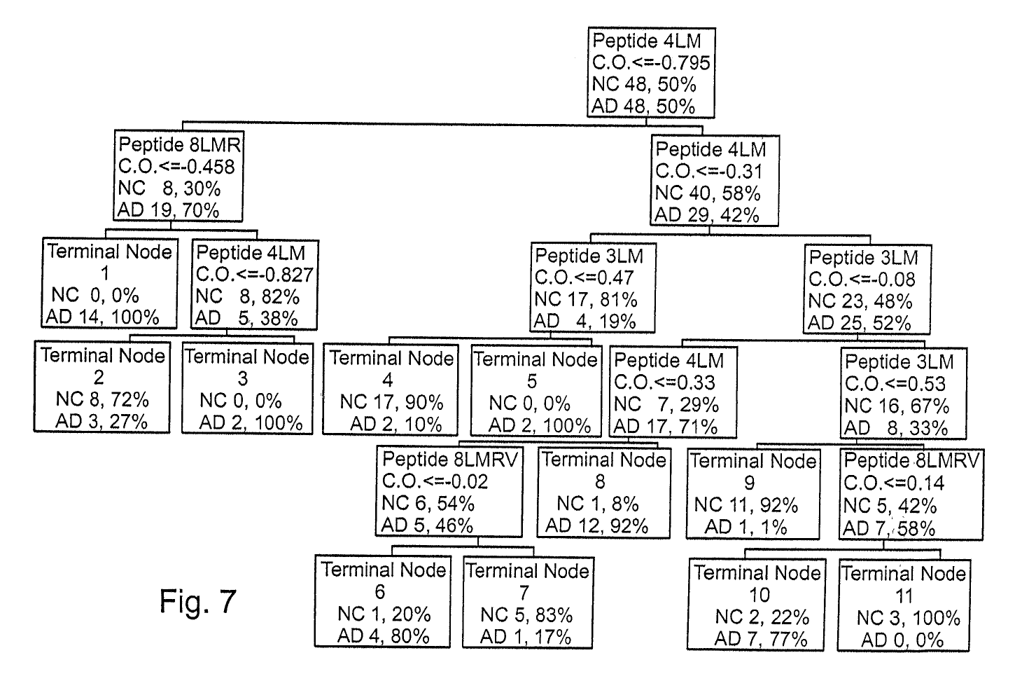

A series of experiments were performed in which 48 AD and 48 NC were analyzed by the use of 17 different peptides (INO; 2L; 2LR; 3NO; 3L; 3M; 3R; 3LM; 3LR; 4LM; 4MR; 4LMR; 5LM; 6LR; 7MR; 8LMR and 8LMR-V). Two different algorithms were developed to analyze the data. The first algorithm included 9 peptides chosen by a computer program according to their relative contribution to the separation between AD and NC sera. In this algorithm the 96 serum samples were separated into two groups according to the value of the signal relative to an empirically set cut-off point. Each group was further analyzed according to the same principle with other or same peptides at certain cut-off points until optimal separation of the (known) AD and NC samples FIG. 7 provides an example for the first algorithm.

The second algorithm first separates the 17 peptides into groups according to the similarities of the profile of signals of the 96 serum samples. As a result, four groups (FACTs) of peptides were defined (Table 4):

TABLE 4

| Peptide | FACT1 | FACT2 | FACT3 | FACT4 |
|---|---|---|---|---|
| 3L | 0.14230 | 0.69548 | 0.27269 | −0.27670 |
| 3M | 0.00926 | 0.69375 | −0.05907 | 0.21262 |
| 3LR | 0.85664 | 0.28486 | 0.08781 | 0.03219 |
| 3LM | 0.02754 | 0.45033 | 0.67562 | −0.17011 |
| 3LM | 0.09140 | 0.83263 | 0.27806 | −0.02242 |
| 4LM | 0.17796 | 0.12518 | 0.81961 | 0.09459 |
| 4MLR | 0.45082 | 0.49102 | 0.24953 | −0.04654 |
| 5LM | 0.38370 | 0.66610 | 0.30991 | −0.10705 |
| 6LR | 0.67788 | 0.24284 | −0.03787 | −0.20259 |
| 7MR | 0.77539 | 0.12649 | 0.07956 | 0.11474 |
| 8LMR | 0.89133 | 0.27983 | 0.10514 | 0.05595 |
| 8MR | 0.74015 | −0.04212 | 0.23881 | 0.17094 |
| 8LMR-V | 0.07948 | 0.02508 | 0.00015 | 0.93403 |
| 2LR | 0.82057 | −0.05813 | 0.07225 | −0.04948 |
| 2L | 0.44146 | 0.70045 | 0.20802 | 0.11253 |
| 4MR | 0.88816 | 0.297820 | 02125 | 0.01311 |

Each peptide within a group is compared to a theoretical “ideal” peptide profile which was given the optimal value of 1. The closer the value calculated for each peptide is to 1, the more similar is the peptide's profile of serum sample signals to the “ideal” peptide profile, and therefor can be regarded as a representative for it's group. According to table 4, therefore, four peptides were identified as representing all 17 peptides: 8LMR; 3LM; 4LM and 8LMR-V.

Using these peptides in an algorithm similar to the one described above (which uses 9 peptides), a scheme separating between AD and NC samples is obtained (See FIG. 7). Summarizing the number of AD and NC samples found in every terminal node results in Tables 5a-b and 6a-b (see below). The terminal nodes are first designated as “AD-nodes” or “NC-nodes” according to what kind of sera is in majority in that node. The serum sample found in minority in such a node is therefore miss-diagnosed. For example, in Table 5a, terminal node No. 4 is designated an “AD-node” since there are 10 AD sera and only one NC serum sample in this node. That single NC serum is a false positive sample. Summarizing the data from Table 5a, Table 5b shows that the 9-peptide algorithm results in a sensitivity of 94% and a specificity of 98%. Similarly, the 4-peptide algorithm results in a sensitivity of 85% and specificity of 92% (Table 6b).

TABLE 5a
Separation of AD and NC in the terminal nodes of the 9-peptide
algorithm.

| Terminal node no. | No. of AD sera* | No. of NC sera* |
|---|---|---|
| 1 2 3 4 5 6 7 8 9 10 11 12 13 14 15 Sum | 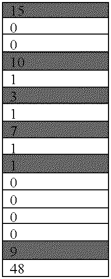 | 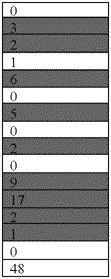 |

*Shaded cell indicates majority of cases in same terminal node.

TABLE 5b
Summary and analysis of data from Table 5a

| | Experimental result = AD | Experimental result = NC |
|---|---|---|
| Indication AD | 45/48 (94% sensitivity) | 2/48 |
| Indication NC | 3/48 | 47/48 (98% specificity) |

TABLE 6a
Separation of AD and NC in the terminal nodes of
the 4-peptide algorithm

| Terminal node no. | No. of AD sera* | No. of NC sera* |
|---|---|---|
| 1 2 3 4 5 6 7 8 9 10 11 Sum | 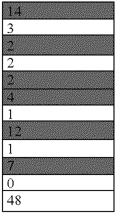 | 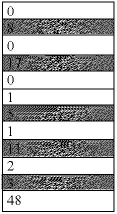 |

*Shaded cell indicates majority of cases in same terminal node

TABLE 6b
Summary and analysis of data from table 4a

| | Experimental result = AD | Experimental result = NC |
|---|---|---|
| Indication AD | 41/48 (85% sensitivity) | 7/48 |
| Indication NC | 4/48 | 44/48 (92% specificity) |

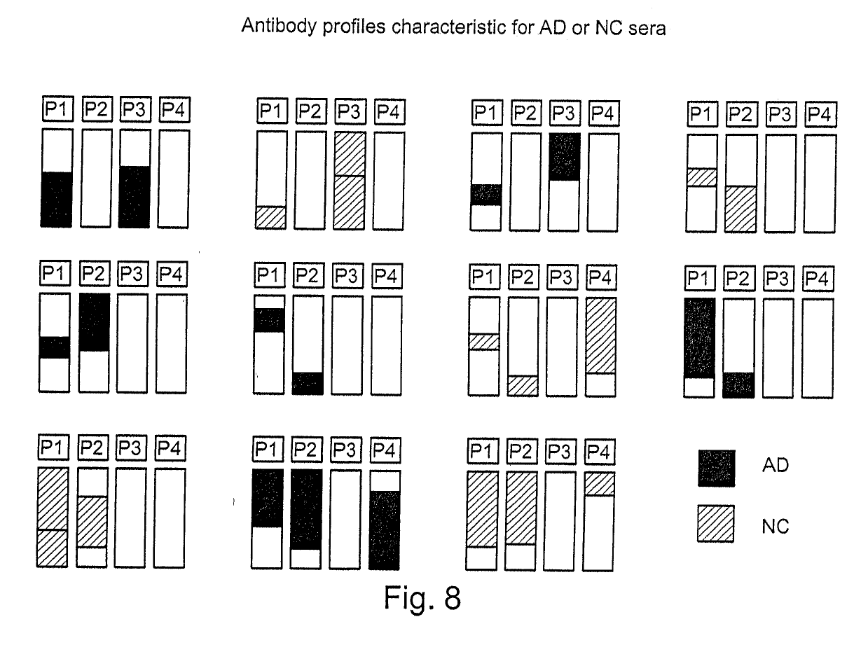

In biochemical terms, the path to the terminal nodes of the four-peptide algorithm (FIG. 7), could be summarized as limited ranges of antibody levels against each of the peptides (FIG. 8). For example, to be defined as AD in terminal node no. 5, the level of antibodies in the sera against peptide 4LM should be above a certain cut-off value, but below another one, and above a cut-off value for peptide 3LM (FIG. 7). Since there are six different characteristic groups defining AD samples, this may be a novel way to define new sub-types of AD. Similarly, the 5 different groups defining NC samples may represent a normal or a pre-pathological variation in the normal population.

Since AD results from a complex syndrome rather than a singular cellular event, the fact that the peptides employed can measure a variety of antibodies, and not a single biochemical marker, will broaden the range of subtypes of AD detectable, and as a result, increase the general sensitivity of the present invention. Prior art methods and kits measure single biochemical markers, and as such cannot detect all the biochemical markers associated with the various AD subtypes.

Thus, the present invention enables to investigate the role of the AD specific antibodies in the etiology of AD. It is likely that the antibodies are not only markers for the presence of the disorder, but rather participate in an autoimmune reaction in the central nervous system. In such a case, the peptides found to have the highest affinity for the AD specific antibodies could be used to bind and remove the antibodies from the blood stream of AD patients. This could potentially stop the progression of the disorder. Thus, the present invention can ultimately lead to a strategy for treating the disorder.

Employing the combination of peptides described above enables the detection of neurodegenerative disorders in general. It will be appreciated that specific sets each including specific combinations of peptides, can be employed for the detection of specific neurodegenerative disorders.

### Example 4

Use of Specific Peptide Combinations for Identifying Specific Disorders

A large set of experiments was performed in efforts to both reduce the number of potentially beneficial peptides and to examine the possibility of using specific peptide subsets for diagnosing various neurodegenerative diseases.

By eliminating peptides which did not contribute to the diagnostic accuracy of the assay, or which generated irreproducible results, the number of potentially useful peptides was reduced from more than 64 to 15. These 15 peptides included: 1LMR-A (SEQ ID NO:21); 2LMR-A (SEQ ID NO:29); 3R (SEQ ID NO:32); 3MR-V (SEQ ID NO:36); 3LMR-V (SEQ ID NO:38); 4LM (SEQ ID NO:42); 4MR (SEQ ID NO:44); 4LMR-A (SEQ ID NO:46); 5LMR-V (SEQ ID NO:54); 6LR (SEQ ID NO:59); 6LMR-V (SEQ ID NO:62); 7MR (SEQ ID NO:68); 7LMR-V (SEQ ID NO:70); 8LMR (SEQ ID NO:77) and 8LMR-V (SEQ ID NO:78).

The above 15 peptides were used to screen 96 Alzheimer's disease (AD) and Normal Control (NC) samples. Classification and Regression Tree (CART) statistics were employed on the results obtained from all possible four peptide combinations of the fifteen peptides utilized. Approximately 1800 algorithms were obtained, of which twelve algorithms (not shown) gave results which were comparable in sensitivity and specificity to that obtained in Example 3 (FIG. 7, Tables 6a and 6b).

Each algorithm was assigned a rank order number which was the sum of the cross-validation specificity rank order and the cross-validation sensitivity rank order of the algorithm. For example, if an algorithm received 2 for specificity and 11 for sensitivity, its overall rank-order would be 13. The lower the rank order number, the more suited the algorithm was for screening.

Cross-validation was performed by constructing the algorithm from 90% of the samples and verifying it against the remaining 10% of the samples.

Seven peptides representing the most suited algorithms were then used to analyze an experimental cassette of samples from four groups of age-matched individuals (see below).

These seven peptides are as follows: 1LMR-A (SEQ ID NO:21); 3R (SEQ ID NO:32); 4LM (SEQ ID NO:42); 5LMR-V (SEQ ID NO:54); 6LR (SEQ ID NO:59); 6LMR-V (SEQ ID NO:62) and 8LMR (SEQ ID NO:77)

Every sample was tested on each of the seven peptides on at least three different days. Four-peptide algorithms were generated from various combination of these seven peptides, and the algorithms were tested for their ability to distinct AD from NC, Multiple Infarct Dementia (MID) or Parkinson's Disease with Dementia (PwD), to distinct NC from MID or PwD, or to distinct MID from PwD.

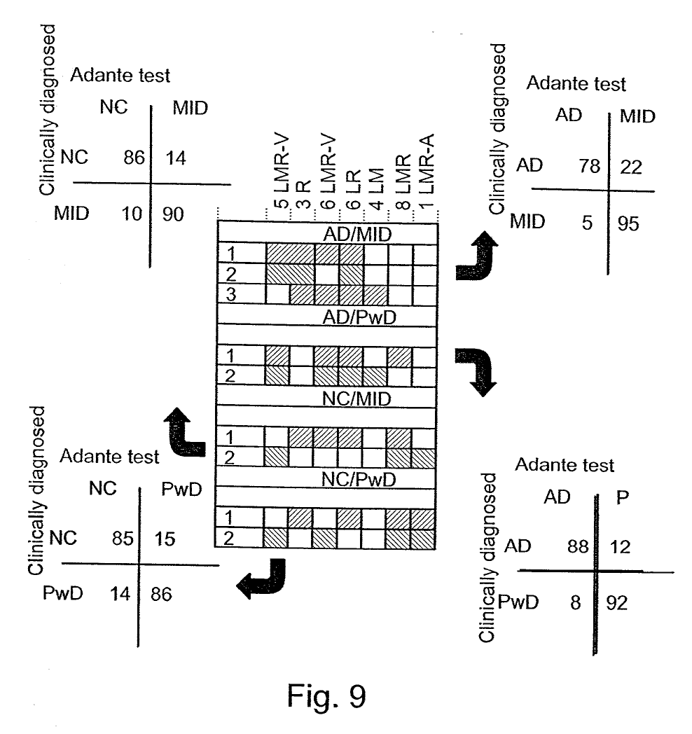

Thirty five CART algorithms were obtained for each of the above comparisons. Results from the most accurate algorithms of four different comparisons are shown in FIG. 9. The best algorithms employed in each comparison test enabled a clear distinction between the two examined groups. The AD/NC comparison is not shown because the results are very similar to those described in Tables 6a and 6b of Example 3. The results from the MID/PwD comparison are also omitted since such a comparison is not diagnostically significant.

From the AD/MID comparison it is clear that the assay of the present invention can be utilized to identify AD (identifying 78% of the clinically diagnosed AD individuals) and MID (95%). Likewise, in the NC/MID comparison, the assay of the present invention provided accurate, identifying 86% of the clinically diagnosed NC individuals and 90% of the clinically diagnosed MID individuals. Thus, use of different combinations of these seven peptides can serve as a tool for specifically distinguishing between NC, AD, MID, and PwD.

Stroke:

Stroke is often preceded by a transient ischemic attack (TIA) caused by a temporary interruption of blood flow to a part of the brain. TIA symptoms (also referred to as a ‘mini-stroke’) are similar to those manifested during a major stroke although TIA symptoms are weaker and of shorter duration. Individuals suffering from TIA are more likely to experience a major stroke [Adams, R D. 1993]; in addition, multiple TIAs can lead to MID.

As described above, various peptide combinations of the present invention enable distinction between NC and MID. Since TIA is an underlying cause for MID and a major risk factor for stroke, it is highly probable that the teachings of the present invention can be used to find a combination of peptides suitable for the detection of TIA.

Recent reports suggest that the level of antibodies against NF rise with time in most stroke victims. In such cases, the screening methodology and peptide combinations of the present invention can be utilized to detect and categorize subgroups of stroke-specific autoantibodies thereby enabling the detection of TIA.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent application or sequence identified by their accession number was specifically and individually indicated to be incorporated herein by reference. In addition, citation or identification of any reference in this application shall not be construed as an admission that such reference is available as prior art to the present invention.

### LIST OF REFERENCES

1. R. Katzman, et al., In: Fields WS, ed. Neurological and sensory disorders in the elderly. New York (1976).

2. J. T. Coyle, et al., Science. 219, 1184-90. Review. (1983).

3. G. e. a. Blessed, Br. J. Psychiatry 114, 797-811 (1968).

4. P. Francis, N. Engl. J. Med. 313, 7-11 (1985).

5. N. R. Sims, et al., J Neurochem. 40, 503-9. (1983).

6. R. T. Bartus, et al., Pharmacol Biochem Behav. 23, 125-35. (1985).

7. G. J. McDougall, Nurse Pract. 15, 18-28. Review. (1990).

8. H. Forstl, J Neural Transm 54, 1-8 (1998).

9. J. Perez-Tur, et al., Neurodegeneration 5, 207-12 (1996).

10. J. Perez-Tur, F. Wavrant-De Vrieze, J. C. Lambert, M. C. Chartier-Harlin, Lancet 347, 1560-1 (1996).

11. J. Perez-Tur, et al., Neuroreport 7, 297-301 (1995).

12. R. Crook, et al., Ann Neurol 42, 124-8 (1997).

13. A. D. Roses, Ann NY Acad Sci. 855, 738-43. Review. (1998).

14. D. Blacker, et al., Nat Genet 19, 357-60 (1998).

15. V. Rudrasingham, et al., Nat Genet 22, 17-9; discussion 21-2 (1999).

16. D. J. Dow, et al., Nat Genet 22, 16-7; discussion 21-2 (1999).

17. F. Wavrant-DeVrieze, et al., Neurosci Lett 262, 137-9 (1999).

18. R. Sherrington, et al., Nature 375, 754-60 (1995).

19. E. Levy-Lahad, et al., Science 269, 973-7 (1995).

20. S. M. de la Monte, L. Volicer, S. L. Hauser, J. R. Wands, Ann Neurol 32, 733-42 (1992).

21. S. M. Monte, et al., J Clin Invest 100, 3093-104 (1997).

22. S. M. De La Monte, R. I. Carlson, N. V. Brown, J. R. Wands, J Neuropathol Exp Neurol 55, 1038-50 (1996).

23. W. A. Jefferies, et al., Brain Res 712, 122-6 (1996).

24. M. L. Kennard, H. Feldman, T. Yamada, W. A. Jefferies, Nat Med 2, 1230-5 (1996).

25. T. Ishii, et al., Acta Neuropathol (Berl). 36, 243-9. (1976).

26. P. Eikelenboom, et al., Acta Neuropathol (Berl). 57, 239-42. (1982).

27. K. Nandy, in Aging: Alzheimer Disease: Senile Dementia and Related Disorders T. R. D. Katzman R., and Bick K. L., Ed. (Raven Press, New York, 1978), vol. 7, pp. 503-511.

28. H. Watts, et al., J Neuroimmunol. 1, 107-16. (1981).

29. H. e. a. Fillit, in Senile dementia of the Alzheimer type. J. T. Hutton, Kenny, A. D., Ed. (Alan R. Liss, New York, 1985) pp. 307-318.

30. J. Rogers, Soc. Neurosci. Abstr. 12, 2596 (1987).

31. A. e. a. Pouplard-Barthelaix, Neurology 37, 225 (1987).

32. P. L. McGeer, et al., Neurosci Lett. 79, 195-200. (1987).

33. D. J. Cameron, Jpn J Exp Med. 55, 177-83. (1985).

34. D. Skias, et al., Neurology. 35, 1635-8. (1985).

35. V. K. Singh, et al., Mech Ageing Dev. 37, 257-64. (1986).

36. J. Chapman, et al., J Neurochem. 51, 479-85. (1988).

37. J. Chapman, et al., J Neurosci. 9, 2710-7. (1989).

38. L. Soussan, et al., Mol Neurobiol. 9, 83-91. (1994).

39. N. H. Sternberger, et al., Proc Natl Acad Sci USA. 82, 4274-6. (1985).

40. I. Grundke-Iqbal, et al., Proc Natl Acad Sci USA. 83, 4913-7. (1986).

41. B. Lichtenberg-Kraag, et al., Proc Natl Acad Sci USA. 89, 5384-8. (1992).

42. E. Masliah, et al., Am J Pathol. 142, 871-82. (1993).

43. L. Soussan, et al., J Neurochem. 62, 770-6. (1994).

44. D. R. Soppet, et al., J Biol Chem. 267, 17354-61. (1992).

45. E. a. S. Atherton, R. C., J. Chem. Soc. Chem. Comm. 165 (1985).

46. R. C. Sheppard, The LKB Journal 33, 9 (1986).

47. M. Bodanszky, Int J Pept Protein Res. 25, 449-74. Review. (1985).

48. D. M. Coe, R. Storer, Mol Divers 4, 31-8 (1998).

49. I. Sucholeiki, Mol Divers 4, 25-30 (1998).

50. F. Albericio, P. Lloyd-Williams, E. Giralt, Methods Enzymol 289, 313-36 (1997).

51. P. A. a. A. Robinson, B. H., Rev. Neurosci. 2, 1-41 (1988).

52. P. M. Steinert, et al., Annu Rev Biochem. 57, 593-625. Review. No abstract available. (1988).

53. J. P. Julien, et al., Biochim Biophys Acta. 755, 25-31. (1983).

54. V. M. -Y. e. a. Lee, Proc. Natl. Adad. Sci USA 185, 1998-2002 (1988).

55. B. A. Wible, et al., Proc Natl Acad Sci USA. 86, 720-4. (1989).

56. H. M. Roder, et al., J Neurosci. 11, 3325-43. (1991).

57. Y. Gonda, et al., Biochem Biophys Res Commun. 167, 1316-25. (1990).

58. R. K. Sihag, et al., J Biol Chem. 265, 4166-71. (1990).

59. T. Tokui, et al., Biochem Biophys Res Commun. 169, 896-904. (1990).

60. A. Dosemeci, et al., Biochem J 282, 477-81. (1992).

61. R. A. Nixon, et al., Trends Neurosci. 14, 501-6. Review. (1991).

62. D. Dahl, Exp Cell Res. 149, 397-408. (1983).

63. N. H. Sternberger, et al., Ann NY Acad Sci. 420, 90-9. (1983).

64. V. M. -Y. e. a. Lee, J. Neurosci. 7, 3474-3488 (1987).

65. D. Dahl, J Neurosci Res. 20, 431-41. (1988).

66. M. J. Carden, et al., J Neurosci. 7, 3489-504. (1987).

67. M. J. Campbell, et al., J Comp Neurol. 282, 191-205. (1989).

68. B. G. Szaro, et al., J Comp Neurol. 302, 220-35. (1990).

69. J. C. Vickers, et al., Neuroscience. 39, 743-59. (1990).

70. A. M. Berglund, et al., J Comp Neurol. 306, 393-408. (1991).

71. E. A. Clark, et al., J Neurosci Res. 30, 116-23. (1991).

72. M. Faigon, et al., J Neurosci Res. 29, 490-8. (1991).

73. M. Goedert, et al., Neuron. 8, 159-68. (1992).

74. M. Goedert, et al., Neuron. 3, 519-26. (1989).

75. Adams R D, Principles of neurology 5th edition. New York: McGraw-Hill, Health Professions Division, 1993.

76. Bornstein, N M et al., submitted.

What is claimed is:

1. A method of identifying an existence, non-existence, type or state of a neurodegenerative disorder in an individual, the method comprising the steps of:
(a) immunoreacting with a serum sample derived from the individual at least one peptide representing at least one epitope derived from an endogenous protein to which at least one antibody is produced in vivo at onset or during progression of the neurodegenerative disorder, said at least one peptide being selected such that said at least one antibody being capable of immunobinding with said at least one peptide; and
(b) detecting a presence, absence or degree of said immunobinding to thereby identify said existence, non-existence, type or state of the neurodegenerative disorder.

2. The method of claim 1, wherein said endogenous protein is selected from the group consisting of NF—H, NF-M, Tau and B-amyloid protein.

3. The method of claim 1, wherein said at least one epitope is a continuous epitope.

4. The method of claim 1, wherein said at least one epitope a discontinuous epitope.

5. The method of claim 1, wherein said at least one peptide includes a number of amino acids selected from the group consisting of at least five, at least six, at least seven, at least eight, at least nine, at least ten, at least eleven, at least twelve, at least thirteen, at least fourteen, at least fifteen, at least sixteen, at least seventeen, between seventeen and twenty five and between twenty five and at least thirty.

6. The method of claim 1, wherein said at least one peptide includes a plurality of peptides and further wherein said at least one antibody includes a plurality of antibodies, whereas said plurality of peptides are selected such that said plurality of antibodies are capable of respectively immunobinding with said plurality of peptides.

7. The method of claim 6, wherein said plurality of peptides are bound in a regiospecific manner to a solid support, such that detecting a presence, absence or degree of said immunobinding to thereby identify said existence, non-existence, type or state of the neurodegenerative disorder is effected by reacting said serum sample with said solid support, identifying reactive peptides according to their regiospecificity and associating said reactive peptides with said existence, non-existence, type or state of the neurodegenerative disorder.

8. The method of claim 1, wherein said at least one peptide includes at least one phospho amino acid.

9. The method of claim 8, wherein said at least one phospho amino acid is selected from the group consisting of phosphoserine, phosphothreonine and phosphotyrosine.

10. The method of claim 9, wherein said phosphoserine forms a part of a sequence motif as set forth in SEQ ID NO:2.

11. The method of claim 9, wherein said phosphoserine forms a part of a sequence motif selected from the group consisting of sequence motives as set forth in SEQ ID NOs: 3, 4 and 5.

12. The method of claim 11, wherein said at least one peptide includes an amino acid sequence selected from the group consisting of SEQ ID NOs:5-76.

13. The method of claim 11, wherein said at least one peptide includes an amino acid sequence as set forth in SEQ ID NO:23.

14. The method of claim 1, wherein the neurodegenerative disorder is associated with progressive loss of cognitive functions.

15. The method of claim 1, wherein the neurodegenerative disorder is associated with progressive loss of control of motoric functions.

16. The method of claim 1, wherein the neurodegenerative disorder is associated with progressive loss of motoric functions.

17. The method of claim 1, wherein the neurodegenerative disorder is selected from the group consisting of Alzheimer's disease, Multi-infarct Dementia (MID), Pick's disease, Frontotemporal dementias, Dementia pugilistica, vascular dementia, Parkinson's disease, Gerstmann-Straussler-Scheinker disease with tangles, Multiple sclerosis, ALS, TIA and stroke.

18. The method of claim 1, wherein said at least one peptide includes an immobilizing moiety covalently attached thereto.

19. The method of claim 1, wherein said immobilizing moiety is a member of a binding pair.

20. The method of claim 19, wherein said member of said binding pair is selected from the group consisting of biotin, avidin, streptavidin, an antibody, a hapten, a receptor, a ligand, Ni and NTA.

21. The method of claim 22, wherein said immobilizing moiety is covalently attached to a terminal of said at least one peptide, said terminal is selected from the group consisting of a carboxy terminal and an amino terminal.

22. The method of claim 1, wherein at least one amino acid of said at least one peptide is a modified amino acid.

23. A proteinaceous substance useful for identifying an existence, non-existence, type or state of a neurodegenerative disorder in an individual, the proteinaceous substance comprising at least one peptide representing at least one epitope derived from an endogenous protein to which at least one antibody is produced in vivo at onset or during progression of the neurodegenerative disorder, said at least one peptide being selected such that said at least one antibody being capable of immunobinding said at least one peptide.

24. The proteinaceous substance of claim 23, wherein said endogenous protein is selected from the group consisting of NF—H, NF-M, Tau and B-amyloid protein.

25. The proteinaceous substance of claim 23, wherein said at least one epitope is a continuous epitope.

26. The proteinaceous substance of claim 23, wherein said at least one epitope a discontinuous epitope.

27. The proteinaceous substance of claim 23, wherein said at least one peptide includes a number of amino acids selected from the group consisting of at least five, at least six, at least seven, at least eight, at least nine, at least ten, at least eleven, at least twelve, at least thirteen, at least fourteen, at least fifteen, at least sixteen and at least seventeen, between seventeen and twenty five and between twenty five and at least thirty.

28. The proteinaceous substance of claim 23, wherein said at least one peptide includes a plurality of peptides and further wherein said at least one antibody includes a plurality of antibodies, whereas said plurality of peptides are selected such that said plurality of antibodies are capable of respectively immunobinding with said plurality of peptides.

29. The proteinaceous substance of claim 23, wherein said at least one peptide includes at least one phospho amino acid.

30. The proteinaceous substance of claim 29, wherein said at least one phospho amino acid is selected from the group consisting of phosphoserine, phosphothreonine and phosphotyrosine.

31. The proteinaceous substance of claim 30, wherein said phosphoserine forms a part of a sequence motif as set forth in SEQ ID NO:2.

32. The proteinaceous substance of claim 30, wherein said phosphoserine forms a part of a sequence motif selected from the group consisting of sequence motives as set forth in SEQ ID NOs: 3, 4 and 5.

33. The proteinaceous substance of claim 23, wherein said at least one peptide includes an amino acid sequence selected from the group consisting of SEQ ID NOs:5-76.

34. The proteinaceous substance of claim 23, wherein said at least one peptide includes an amino acid sequence as set forth in SEQ ID NO:23.

35. The proteinaceous substance of claim 23, wherein the neurodegenerative disorder is associated with progressive loss of cognitive functions.

36. The proteinaceous substance of claim 23, wherein the neurodegenerative disorder is associated with progressive loss of control of motoric functions.

37. The proteinaceous substance of claim 23, wherein the neurodegenerative disorder is associated with progressive loss of motoric functions.

38. The proteinaceous substance of claim 23, wherein the neurodegenerative disorder is selected from the group consisting of Alzheimer's disease, Multi-infarct Dementia (MID), Pick's disease, Frontotemporal dementias, Dementia pugilistica, vascular dementia, Parkinson^{t}s disease, Gerstmann-Straussler-Scheinker disease with tangles, Multiple sclerosis, ALS, TIA and stroke.

39. The proteinaceous substance of claim 23, wherein said at least one peptide includes an immobilizing moiety covalently attached thereto.

40. The proteinaceous substance of claim 23, wherein said immobilizing moiety is a member of a binding pair.

41. The proteinaceous substance of claim 40, wherein said member of a binding pair is selected from the group consisting of biotin, avidin, streptavidin, an antibody, a hapten, a receptor, a ligand, Ni and NTA.

42. The proteinaceous substance of claim 40, wherein said immobilizing moiety is covalently attached to a terminal of said at least one peptide, said terminal is selected from the group consisting of a carboxy terminal and an amino terminal.

43. The proteinaceous substance of claim 23, wherein at least one amino acid of said at least one peptide is a modified amino acid.

44. The proteinaceous substance of claim 23, further comprising a display polypeptide covalently attached to said at least one peptide at a terminal thereof, said terminal is selected from the group consisting of an amino terminal and a carboxy terminal.

45. The proteinaceous substance of claim 44, wherein said display polypeptide forms a part of a display system selected from the group consisting of a phage display system and a bacterial display system.

46. A filter for removing at least one antibody generated against an endogenous protein associated with the onset or progression of the neurodegenerative disorder from the blood of a patient suffering from the neurodegenerative disorder, said filter comprising a solid support and the proteinaceous substance of claim 23 attached thereto such that filtering the blood of a patient suffering from the neurodegenerative disorder through said filter substantially removes the at least one antibody therefrom.

47. An extracorporeal device for removing at least one antibody generated against an endogenous protein associated with the onset or progression of a neurodegenerative disorder from the blood of a patient suffering from the neurodegenerative disorder, the extracorporeal device comprising:
(a) the filter of claim 46; and
(b) a pump for circulating the blood of the patient suffering from the neurodegenerative disorder through said filter, such that the at least one antibody is substantially removed from the blood of a patient.

48. A peptide comprising an amino acid sequence representing at least one epitope of an endogenous protein to which at least one antibody is produced in vivo at onset or during progression of a neurodegenerative disorder.

49. The peptide of claim 48, wherein said epitope is selected from the group consisting of a continuous epitope and a discontinuous epitope.

50. The peptide of claim 48, wherein said amino acid sequence includes at least one phospho amino acid.

51. The peptide of claim 50, wherein said at least one phospho amino acid is selected from the group consisting of phosphoserine, phosphothreonine and phosphotyrosine.

52. The peptide of claim 48, wherein said amino acid sequence includes at least one modified amino acid.

53. The peptide of claim 48, wherein said endogenous protein is selected from the group consisting of NF—H NF-M and Tau.

54. The peptide of claim 48, selected from the group consisting of SEQ ID NOs:5-76.

55. A method of identifying peptides useful for identifying an existence, non-existence, type or state of a neurodegenerative disorder in an individual, the method comprising the steps of:
(a) preparing a plurality of peptides corresponding to a plurality of continuous or discontinuous sequences derived from an endogenous protein to which at least one antibody is produced in vivo at onset or during progression of the neurodegenerative disorder;
(b) screening said plurality of peptides for at least one peptide being immunoreactive with a serum derived from at least one patient suffering from the neurodegenerative disorder, thereby identifying peptides useful of identifying an existence, non-existence, type or state of the neurodegenerative disorder

56. The method of claim 55, wherein said continuous or discontinuous sequences derived from said endogenous protein include at least one phospho amino acid.

57. The method of claim 56, wherein said at least one phospho amino acid is selected from the group consisting of phosphoserine, phosphothreonine and phosphotyrosine.

58. The method of claim 55, wherein said continuous or discontinuous sequences derived from said endogenous protein include at least one repeat of the sequence set forth by SEQ ID NO:2.

59. The method of claim 55, wherein said continuous or discontinuous sequences derived from said endogenous protein include at least one sequence motif selected from the group consisting of SEQ ID NOs:1, 3 and 4.

60. The method of claim 55, wherein said step of preparing said plurality of peptides includes covalently attaching to each of said plurality of peptides at least one immobilizing moiety.

61. The method of claim 60, wherein said immobilizing moiety is a member of a binding pair.

62. The method of claim 61, wherein said member of said binding pair is selected from the group consisting of biotin, avidin, streptavidin, an antibody, a hapten, a receptor, a ligand, Ni and NTA.

63. The method of claim 61, wherein said immobilizing moiety is covalently attached to a terminal of said at least one peptide, said terminal is selected from the group consisting of a carboxy terminal and an amino terminal.

64. The method of claim 61, wherein said at least one peptide includes at least one modified amino acid.

65. A method of removing at least one antibody generated against an endogenous protein associated with the onset or progression of a neurodegenerative disorder from the blood of a patient suffering from the neurodegenerative disorder, the method comprising the step of circulating the blood of the patient through an extracorporeal device including at least one peptide representing at least one epitope derived from an endogenous protein and capable of immunobinding at least one antibody recognizing said endogenous protein and which is associated with said neurodegenerative disorder, said extracorporeal device is configured such that when the blood of the patient is circulated therethrough said at least one peptide immunobinds said at least one antibody to thereby substantially remove antibodies associated with the neurodegenerative disorder from the blood of the patient.

66. The method of claim 65, wherein said endogenous protein is selected from the group consisting of NF—H, NF-M, Tau and B-amyloid protein.

67. The method of claim 65, wherein said at least one epitope is a continuous epitope.

68. The method of claim 65, wherein said at least one epitope a discontinuous epitope.

69. The method of claim 65, wherein said at least one peptide includes a number of amino acids selected from the group consisting of at least five, at least six, at least seven, at least eight, at least nine, at least ten, at least eleven, at least twelve, at least thirteen, at least fourteen, at least fifteen, at least sixteen and at least seventeen, between seventeen and twenty five and between twenty five and at least thirty.

70. The method of claim 65, wherein said at least one peptide includes a plurality of peptides and further wherein said at least one antibody includes a plurality of antibodies, whereas said plurality of peptides are selected such that said plurality of antibodies are capable of respectively immunobinding with said plurality of peptides.

71. The method of claim 65, wherein said at least one peptide includes at least one phospho amino acid.

72. The method of claim 71, wherein said at least one phospho amino acid is selected from the group consisting of phosphoserine, phosphothreonine and phosphotyrosine.

73. The method of claim 72, wherein said phosphoserine forms a part of a sequence motif as set forth in SEQ ID NO:2.

74. The method of claim 72, wherein said phosphoserine forms a part of a sequence motif selected from the group consisting of sequence motives as set forth in SEQ ID NOs: 3, 4 and 5.

75. The method of claim 73, wherein said at least one peptide includes an amino acid sequence selected from the group consisting of SEQ ID NOs:5-76.

76. The method of claim 73, wherein said at least one peptide includes an amino acid sequence as set forth in SEQ ID NO:23.

77. The method of claim 65, wherein the neurodegenerative disorder is associated with progressive loss of cognitive functions.

78. The method of claim 65, wherein the neurodegenerative disorder is associated with progressive loss of control of motoric functions.

79. The method of claim 65, wherein the neurodegenerative disorder is associated with progressive loss of motoric functions.

80. The method of claim 65, wherein the neurodegenerative disorder is selected from the group consisting of Alzheimer's disease, Multi-infarct Dementia (MID), Pick's disease, Frontotemporal dementias, Dementia pugilistica, vascular dementia, Parkinson's disease, Gerstmann-Straussler-Scheinker disease with tangles, Multiple sclerosis, ALS, TIA and stroke.

81. The method of claim 65, wherein said at least one peptide includes an immobilizing moiety covalently attached thereto.

82. The method of claim 81, wherein said immobilizing moiety is a member of a binding pair.

83. The method of claim 82, wherein said member of a binding pair is selected from the group consisting of biotin, avidin, streptavidin, an antibody, a hapten, a receptor, a ligand, Ni and NTA.

84. The method of claim 81, wherein said immobilizing moiety is covalently attached to a terminal of said at least one peptide, said terminal is selected from the group consisting of a carboxy terminal and an amino terminal.

85. The method of claim 65, wherein at least one amino acid of said at least one peptide is a modified amino acid.

86. the method of claim 65, wherein the extracorporeal device includes a pump for circulating the blood of the patient through the extracorporeal device.

87. An array device useful for identifying an existence, non-existence, type or state of a neurodegenerative disorder in an individual, the array device comprising a plurality of peptides each being attached to a solid support in a regiospecific manner, said plurality of peptides representing epitopes derived from at least one endogenous protein to which a plurality of antibodies are produced in vivo at onset or during progression of the neurodegenerative disorder, each of said plurality of peptides being selected such that each of said plurality of antibodies being capable of immunobinding said at least each of said plurality of peptides.

88. The array device of claim 87, wherein said at least one endogenous protein is selected from the group consisting of NF—H, NF-M, Tau and B-amyloid protein.

89. The array device of claim 87, wherein said at least one epitope is a continuous epitope.

90. The array device of claim 87, wherein said at least one epitope a discontinuous epitope.

91. The array device of claim 87, wherein said each of a plurality of peptides includes a number of amino acids selected from the group consisting of at least five, at least six, at least seven, at least eight, at least nine, at least ten, at least eleven, at least twelve, at least thirteen, at least fourteen, at least fifteen, at least sixteen and at least seventeen, between seventeen and twenty five and between twenty five and at least thirty.

92. The array device of claim 87, wherein each of said plurality of peptides includes at least one phospho amino acid.

93. The array device of claim 92, wherein said at least one phospho amino acid is selected from the group consisting of phosphoserine, phosphothreonine and phosphotyrosine.

94. The array of claim 93, wherein said phosphoserine forms a part of a sequence motif as set forth in SEQ ID NO:2.

95. The array of claim 93, wherein said phosphoserine forms a part of a sequence motif selected from the group consisting of sequence motives as set forth in SEQ ID NOs: 3, 4 and 5.

96. The array device of claim 94, wherein each of said plurality of peptides includes an amino acid sequence selected from the group consisting of SEQ ID NOs:5-76.

97. The array device of claim 94, wherein each of said plurality of peptides is of an amino acid sequence selected from the group consisting of SEQ ID NOs: 21, 29, 32, 36, 38, 42, 44, 46, 54, 59, 62, 68, 70, 77 and 78.

98. The array device of claim 94, wherein each of said plurality of peptides is of an amino acid sequence selected from the group consisting of SEQ ID NOs: 21, 32, 42, 54, 59, 62 and 77.

99. The array device of claim 87, wherein the neurodegenerative disorder is associated with progressive loss of cognitive functions.

100. The array device of claim 87, wherein the neurodegenerative disorder is associated with progressive loss of control of motoric functions.

101. The array device of claim 87, wherein the neurodegenerative disorder is associated with progressive loss of motoric functions.

102. The array device of claim 87, wherein the neurodegenerative disorder is selected from the group consisting of Alzheimer's disease, Multi-infarct Dementia (MID), Pick's disease, Frontotemporal dementias, Dementia pugilistica, vascular dementia, Parkinson's disease, Gerstmann-Straussler-Scheinker disease with tangles, Multiple sclerosis, ALS, TIA and stroke.

103. The array device of claim 87, wherein each of said plurality of peptides is attached to the solid support via an immobilizer.

104. The array device of claim 87, wherein said immobilizer includes a first member and a second member of a binding pair.

105. The array of claim 104, wherein said first member is covalently attached to each of said plurality of peptides and said second member is covalently attached to said solid support.

106. The array device of claim 104, wherein said first and second members of said binding pair are each independently selected from the group consisting of biotin, avidin, streptavidin, an antibody, a hapten, a receptor, a ligand, Ni and NTA.

107. The array device of claim 104, wherein said first member is a moiety covalently attached to a terminal of said each of said plurality of peptides, said terminal is selected from the group consisting of a carboxy terminal and an amino terminal.

108. The array device of claim 87, wherein at least one amino acid of said at least one peptide is a modified amino acid.

109. A method of generating a peptide combination useful for identifying an existence, non-existence, type or state of a neurodegenerative disorder in an individual, the method comprising the steps of:
(a) identifying at least one endogenous protein to which at least one antibody is produced in vivo at onset or during progression of the neurodegenerative disorder;
(b) generating a plurality of peptides corresponding to said at least one endogenous protein;
(c) reacting specific subsets of said plurality of peptide with serum obtained from:
(i) a first population of individuals suffering from the neurodegenerative disorder; and
(ii) a second population of individuals not suffering from the neurodegenerative disorder; and
(d) identifying specific subset or subsets of the plurality of peptides being immunoreactive with a high number of said individuals of said first population and a low number of said individuals of said second population to thereby generate the peptide combination useful for identifying an existence, non-existence, type or state of a neurodegenerative disorder in an individual.

110. the method of claim 109, wherein said plurality of peptides are overlapping peptides.

111. The method of claim 109, wherein each of said p[plurality of peptides includes a number of amino acids selected from the group consisting of at least five, at least six, at least seven, at least eight, at least nine, at least ten, at least eleven, at least twelve, at least thirteen, at least fourteen, at least fifteen, at least sixteen, at least seventeen, between seventeen and twenty five and between twenty five and at least thirty.

112. The method of claim 109, wherein said plurality of peptides are bound in a regiospecific manner to a solid support, such that reactive peptides are identifiable according to their regiospecificity.

113. The method of claim 109, wherein at least a portion of said plurality of peptides each include at least one phospho amino acid.

114. The method of claim 109, wherein said at least one phospho amino acid is selected from the group consisting of phosphoserine, phosphothreonine and phosphotyrosine.

115. A method of identifying an existence, non-existence, type or state of a neurodegenerative disorder in an individual, the method comprising the steps of:
(a) immunoreacting a serum sample derived from the individual with a plurality of peptides, each peptide of said plurality of peptides representing at least one epitope derived from an endogenous protein to which at least one antibody is produced in vivo at onset or during progression of the neurodegenerative disorder; and
(b) detecting a presence, absence or degree of antibody binding to each of said plurality of peptides to thereby generate an immunobinding profile for said serum sample derived from the individual, said profile being indicative of the existence, non-existence, type or state of the neurodegenerative disorder.

116. The method of claim 115, wherein said endogenous protein is selected from the group consisting of NF—H, NF-M, Tau and B-amyloid protein.

117. The method of claim 115, wherein the neurodegenerative disorder is selected from the group consisting of Alzheimer's disease, Multi-infarct Dementia (MID), Pick's disease, Frontotemporal dementias, Dementia pugilistica, vascular dementia, Parkinson's disease, Gerstmann-Straussler-Scheinker disease with tangles, Multiple sclerosis, ALS, TIA and stroke.

118. The method of claim 115, wherein said plurality of peptides are bound in a regiospecific manner to a solid support, such that said immunobinding profile is generated by identifying reactive peptides of said plurality of peptides according to their regiospecificity.

119. The method of claim 115, wherein each peptide of said plurality of peptides includes at least one phospho amino acid.

120. The method of claim 119, wherein said at least one phospho amino acid is selected from the group consisting of phosphoserine, phosphothreonine and phosphotyrosine.

121. The method of claim 120, wherein said phosphoserine forms a part of a sequence motif as set forth in SEQ ID NO:2.

122. The method of claim 120, wherein said phosphoserine forms a part of a sequence motif selected from the group consisting of sequence motives as set forth in SEQ ID NOs: 3, 4 and 5.

123. The method of claim 115, wherein said plurality of peptides are selected from the group of peptides set forth in SEQ ID NOs:5-76.

124. The method of claim 115, wherein said plurality of peptides are selected from the group of peptides set forth in SEQ ID NOs: 21, 29, 32, 36, 38, 42, 44, 46, 54, 59, 62, 68, 70, 77 and 78.

125. The method of claim 115, wherein said plurality of peptides are selected from the group of peptides set forth in SEQ ID NOs: 21, 32, 42, 54, 59, 62 and 77.

126. The method of claim 115, wherein the neurodegenerative disorder is associated with progressive loss of cognitive functions.

127. The method of claim 115, wherein the neurodegenerative disorder is associated with progressive loss of control of motoric functions.

128. The method of claim 115, wherein the neurodegenerative disorder is associated with progressive loss of motoric functions.

129. The method of claim 115, wherein each peptide of said plurality of peptides includes an immobilizing moiety covalently attached thereto.

130. A method of predicting the presence of a neurodegenerative disorder in a subject, the method comprising the steps of:
(a) immunoreacting a sample derived from the subject with a plurality of peptides so as to form a complex, wherein each peptide of said plurality of peptides represents at least one epitope derived from an endogenous protein to which at least one antibody is produced during progression of the neurodegenerative disorder;
(b) detecting said complex, thereby generating an immunobinding profile for said sample derived from the subject; and
(c) comparing said immunoblotting profile of said sample to a normative value thereby predicting the presence of the neurodegenerative disorder in the subject.

131. The method of claim 130, wherein said endogenous protein is selected from the group consisting of NF—H, NF-M, and Tau.

132. The method of claim 130, wherein the neurodegenerative disorder is selected from the group consisting of, Pick's disease, Frontotemporal dementias, Dementia pugilistica, vascular dementia, Parkinson's disease, Gerstmann-Straussler-Scheinker disease with tangles, Multiple sclerosis, and ALS.

133. The method of claim 130, wherein the neurodegenerative disease is Alzheimer's disease.

134. The method of claim 130, wherein said sample is a blood sample.

135. The method of claim 130, wherein each peptide of said plurality of peptides includes at least one phospho amino acid.

136. The method of claim 135, wherein said at least one phospho amino acid is phosphoserine, phosphothreonine or phosphotyrosine.

137. The method of claim 136, wherein said phosphoserine is a part of at least one repeated sequence motif as set forth in SEQ ID NO:2.

138. The method of claim 136, wherein said phosphoserine is a part of at least one repeated sequence motif selected the sequence motives as set forth in SEQ ID NOs: 3, 4 and 5.

139. The method of claim 130, wherein said plurality of peptides are selected from the group of peptides set forth in SEQ ID NOs:5-78.

140. The method of claim 130, wherein said plurality of peptides are selected from the group of peptides set forth in SEQ ID NOs: 21, 29, 32, 36, 38, 42, 44, 46, 54, 59, 62, 68, 70, 77 and 78.

141. The method of claim 130, wherein said plurality of peptides are selected from the group of peptides set forth in SEQ ID NOs: 21, 32, 42, 54, 59, 62 and 77.

142. The method of claim 130, wherein each peptide of said plurality of peptides includes an immobilizing moiety covalently attached thereto.

143. A method of predicting the state of a neurodegenerative disorder in a subject, the method comprising the steps of:
(a) immunoreacting a sample derived from the subject with a plurality of peptides so as to form a complex so as to form a complex, wherein each peptide of said plurality of peptides represents at least one epitope derived from an endogenous protein to which at least one antibody is produced during progression of the neurodegenerative disorder;
(b) detecting said complex, thereby generating an immunobinding profile for said sample derived from the subject; and
(c) comparing said immunobinding profile of said sample to a normative value thereby predicting the state of the neurodegenerative disorder in the subject.

144. The method of claim 143, wherein said endogenous protein is selected from the group consisting of NF—H, NF-M, and Tau.

145. The method of claim 143, wherein the neurodegenerative disorder is selected from the group consisting of, Pick's disease, Frontotemporal dementias, Dementia pugilistica, vascular dementia, Parkinson's disease, Gerstmann-Straussler-Scheinker disease with tangles, Multiple sclerosis, and ALS.

146. The method of claim 143, wherein the neurodegenerative disease is Alzheimer's disease.

147. The method of claim 143, wherein said sample is a blood sample.

148. The method of claim 143, wherein each peptide of said plurality of peptides includes at least one phospho amino acid.

149. The method of claim 148, wherein said at least one phospho amino acid is phosphoserine, phosphothreonine or phosphotyrosine.

150. The method of claim 149, wherein said phosphoserine is a part of at least one repeated sequence motif as set forth in SEQ ID NO:2.

151. The method of claim 149, wherein said phosphoserine is a part of at least one repeated sequence motif selected the sequence motives as set forth in SEQ ID NOs: 3, 4 and 5.

152. The method of claim 143, wherein said plurality of peptides are selected from the group of peptides set forth in SEQ ID NOs:5-78.

153. The method of claim 143, wherein said plurality of peptides are selected from the group of peptides set forth in SEQ ID NOs: 21, 29, 32, 36, 38, 42, 44, 46, 54, 59, 62, 68, 70, 77 and 78.

154. The method of claim 143, wherein said plurality of peptides are selected from the group of peptides set forth in SEQ ID NOs: 21, 32, 42, 54, 59, 62 and 77.

155. The method of claim 143, wherein each peptide of said plurality of peptides includes an immobilizing moiety covalently attached thereto.

156. A method of predicting the presence of an ischemic disorder in a subject, comprising the steps of:
(a) immunoreacting a sample derived from the subject with a plurality of peptides so as to form a complex, wherein each peptide of said plurality of peptides represents at least one epitope derived from an endogenous protein to which at least one antibody is produced during progression during progression of the ischemic disorder
(b) detecting said complex, thereby generating an immunobinding profile for said sample derived from the subject; and
(c) comparing said immunobinding profile of said sample to a normative value thereby of predicting the presence of an ischemic disorder in a subject.

157. The method of claim 156, wherein said endogenous protein is selected from the group consisting of NF—H, NF-M and Tau.

158. The method of claim 156, wherein the ischemic disorder is selected from the group consisting of stroke, Transient Ischemic Attack (TIA) and Multiple Infract Dementia (MID).

159. The method of claim 156, wherein each peptide of said plurality of peptides includes at least one phospho amino acid.

160. The method of claim 159, wherein said at least one phospho amino acid is selected from the group consisting of phosphoserine, phosphothreonine and phosphotyrosine.

161. The method of claim 160, wherein said phosphoserine is a part of at least one repeated sequence motif as set forth in SEQ ID NO:2.

162. The method of claim 160, wherein said phosphoserine is a part of at least one repeated sequence motif selected the sequence motives as set forth in SEQ ID NOs: 3, 4 and 5.

163. The method of claim 156, wherein said plurality of peptides are selected from the group of peptides set forth in SEQ ID NOs:5-78.

164. The method of claim 156, wherein said plurality of peptides are selected from the group of peptides set forth in SEQ ID NOs: 21, 29, 32, 36, 38, 42, 44, 46, 54, 59, 62, 68, 70, 77 and 78.

165. The method of claim 156, wherein said plurality of peptides are selected from the group of peptides set forth in SEQ ID NOs: 21, 32, 42, 54, 59, 62 and 77.

166. The method of claim 156, wherein each peptide of said plurality of peptides includes an immobilizing moiety covalently attached thereto.
